(19) United States
(12) Patent Application Publication
(10) Pub. No.: US 2011/0028552 A1
(43) Pub. Date: Feb. 3, 2011

(54) MEXILETINE AMINO ACID AND PEPTIDE PRODRUGS AND USES THEREOF

(76) Inventors: Richard Franklin, Hampshire (GB); Bernard T. Golding, Newcastle upon Tyne (GB); Robert G. Tyson, Durham (GB)

Correspondence Address: MCDERMOTT WILL & EMERY LLP, 600 13TH STREET, N.W., WASHINGTON, DC, 20005-3096, US

(21) Appl. No.: 12/823,066

(22) Filed: Jun. 24, 2010

Related U.S. Application Data
(60) Provisional application No. 61/269,458, filed on Jun. 24, 2009.

(51) Int. Cl. A61K 31/197; C07C 237/08; A61K 31/165; C07C 271/54; A61P 29/00; A61P 9/06

(52) U.S. Cl. 514/563; 564/196; 514/626; 562/448; 564/157; 514/616; 558/271; 560/164

(57) ABSTRACT

The present invention concerns prodrugs of mexiletine (and mexiletine's active metabolite) with amino acids or peptides and pharmaceutical compositions containing such prodrugs. Methods for providing pain relief, treating arrhythmia, decreasing the adverse GI side effects associated with mexiletine, increasing the bioavailability of mexiletine, and improving the pharmacokinetic reproducibility of mexiletine with the aforementioned prodrugs are also provided. Oligopeptides incorporating lysine or arginine residues attached directly or indirectly through a glycine residue are also described herein.

### CROSS-REFERENCE TO RELATED APPLICATIONS

This application claims benefit under 35 U.S.C. §119(e) to U.S. Provisional Application No. 61/269,458 filed on Jun. 24, 2009, which is hereby incorporated by reference in its entirety.

### FIELD OF THE INVENTION

The present invention relates to various amino acid and peptide prodrugs of mexiletine, and the use of the prodrugs to improve mexiletine's pharmacokinetic consistency, to treat neuropathic pain, and to avoid the adverse gastrointestinal (GI) side effects commonly associated with mexiletine. It may also be used for treating arrhythmias.

### BACKGROUND OF THE INVENTION

Neuropathic pain is estimated to impact between 2.8 and 4.7% of the global population (Neuropathic Pain Network and Pfizer Inc., 2006 survey). Broadly classified as central or peripheral, neuropathic pain is caused by injury to, or disease of, the nervous system, or pain derived from damage to the nervous system itself, rather than pain detected by the nervous system due to external stimuli such as burns or broken limbs. Central neuropathic pain occurs as a result of damage to the central nervous system (CNS), and can be caused by, for example, multiple sclerosis, spinal cord injury, stroke or cancer. Peripheral neuropathic pain arises from damage to the peripheral nervous system caused by diabetes, cancer, HIV infection, carpel tunnel syndrome and post hepatic neuralgia, amputation (phantom limb pain), back injury, leg ulcers and iatrogenic injury through surgery. Across the seven major pharmaceutical markets a recent report estimated that around 37.6 million patients suffer from central neuropathic pain while some 170 million suffer from peripheral neuropathic pain (Neuropathic Pain Network and Pfizer Inc, 2006 survey).

Symptoms of neuropathic pain include a burning, shooting, stabbing or electric shock type sensations. Other common neuropathic pain symptoms are allodynia (pain due to normally non-painful stimuli), hyperesthesia (an exaggerated response to light touch) and hyperpathy (persistent pain even after the cause of the pain is removed) and dysthesia (abnormal and unpleasant tingling or pins and needles sensation).

Neuropathic pain is more common in certain patient populations. For example, up to a quarter of diabetic patients and a third of cancer patients experience such pain. Furthermore, over half of patients suffering from shingles develop post herpetic neuralgia and a third of patients with spinal injury are affected by neuropathic pain (Neuropathic Pain Network and Pfizer Inc, 2007 survey).

Currently, there are few effective treatments for neuropathic pain. Pregabalin, gabapentin, duloxetine (a serotonin-norepinephrine reuptake inhibitor (SNRI) anti-depressant), Δ9 tetrahydrocannibinol and lidocaine patches (for local treatment of post herpetic neuralgia) are amongst the currently available treatment options. Each, however, has its own distinct limitations. For example, pregabalin is associated with significant adverse CNS effects. The side effects most frequently leading to pregabalin discontinuation were dizziness and somnolence. These two side effects occurred in up to 30% of patients treated at the higher doses of pregabalin (FDA labeling). In the case of gabapentin, its oral bioavailability is not proportional to dose i.e., as dose is increased, bioavailability decreases. Bioavailabilities of approximately 60%, 47%, 34%, 33%, and 27% were observed following 900, 1200, 2400, 3600, and 4800 mg/day gabapentin (FDA labeling). Duloxetine is associated with nausea in 20-40% of treated patients, as well as suicidality concerns in treated patients (FDA labeling). Δ9 tetrahydrocannibinol has a distinct addiction liability (DEA classification).

Mexiletine, (rac)-1-(2,6-dimethylphenoxy)-2-propanamine hydrochloride (structure shown below) is a sodium channel blocking agent which as a consequence has local anesthetic properties (Scholz A (2002) Brit. J Anaethesia 89, 52-61). Mexiletine first found utility as a Class 1B anti-arrhythmic agent, and is still used today to treat arrhythmias. The drug is currently available as 150 mg, 200 mg or 250 mg capsules and is indicated for the treatment of ventricular arrhythmias. The most frequent adverse reaction associated with mexiletine administration is upper gastrointestinal distress (FDA label).

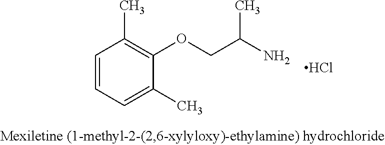

In more recent years, mexiletine has found increasing utility in the treatment of neuropathic pain of various origins. Its use has been reported for diabetic neuropathy, acute and chronic nerve pain, alcoholic polyneuropathy, chronic pain from radiotherapy, thalamic pain and diabetic truncal pain (Jarvis and Coukell (1998). Drugs 4, 691-707). Additionally more recent reports suggest the utility of mexiletine in the treatment of erythromelaglia (EM), a rare disabling disorder characterized by recurrent burning pain, erythema, and increased temperature of the affected areas (e.g., feet and ears). (Vivas A C et al (2010) Amer. J. Otolaryngology, May). Additionally mexiletine has been found to be useful in chronic cryptogenic sensory polyneuropathy, a condition in which patients present with numbness or tingling in the distal lower extremities (Wolfe G I et al (1999) Arch Neurol 56 540-547).

However, the use mexiletine is accompanied by a high incidence of nausea, vomiting and abdominal discomfort (38% of treated patients) (Morganroth (1987). Am. J. Cardiol. 60, 1276-1281). Such adverse GI side effects undoubtedly contribute to poor patient compliance. Emesis will also result in partial loss of the administered drug and consequently, a reduced and unpredictable efficacy. Emesis can be a dose limiting side-effect of oral mexiletine and may preclude attainment of effective plasma drug concentrations. (Wright et al. (1997). Ann Pharmacother. 31, 29-34 and Galer et al. (1996). J Pain Symptom Manage. 12, 161-167).

There is only limited understanding of the mechanism of mexiletine's emetic action. One experimental study has shown that mexiletine can decrease the slow-wave activity in the rat stomach in vivo, but had no effect on jejeunal myoelectrical activity (Bielefeldt and Bass (1991). Digestion 48, 43-50). Inhibition of gastric slow wave activity is considered to play a key role in the induction of nausea and vomiting. In the case of mexiletine the inhibition of gastric slow wave activity may be effected through its local anaesthetic (sodium channel blocking) activity. Other in vitro work using the rabbit oesophageal sphincter suggested that mexiletine, like the intravenous anesthetic compounds ketamine and midazolam, may inhibit the non-adrenergic, non-cholinergic (NANC) relaxation brought out by nitric oxide (Kohjitani et al. (2003). Eur. J. Pharmacol., 465, 145-151). This study concluded that suppression of endogenous nitric oxide in the lower oesophageal sphincter smooth muscle by mexiletine may contribute to the adverse GI effects of mexiletine.

Studies conducted on another local anaesthetic agent lignocaine point to the emetic effects associated with oral administration of that compound being induced by a direct action on the gut. After equi effective iv and po anti-arrhythmic doses of the drug given to dogs only the orally administered drug induced emesis despite comparable systemic blood levels being reached in each. (Smith E R et al 1972) Amer. Heart Journal 83 363-372)

There are also reports in the literature which suggest that mexiletine may have inherent gastric irritant properties. For example periodic cases of oesphagistis following mexiletine ingestion have been reported (Penalba C (1986) Ann Gastroenterol Hepatol (Pris) 22, 267-268, Seggewiss R R & Seckfort H (1983) Dtsch Med. Wochenshr. 108 1018-1020, Addler J B (1990) Am J Gastroenterol. 85 629-630). Thus it is possible that the emetic effects of mexiletine could more simply be due to a direct irritant effect on the stomach.

In spite of such advances in understanding of the mechanism of these adverse events, there continues to be a need to reduce side-effects associated with mexiletine therapy. There remains therefore a real need in the treatment of pain for a mexiletine product which retains all the inherent pharmacological advantages of the drug molecule but overcomes its limitations in inducing adverse GI side-effects. The present invention addresses this need.

Although efficacy and toxicity are important considerations when administering any pharmaceutical compound, in the case of mexiletine the emetic properties are actually a greater barrier to patient compliance and to adequate and therapeutically effective dosing levels. Consequently, the benefits provided by the compounds of the invention in reducing or eliminating emesis when treating with mexiletine are expected to be significant. The invention will thus provide easy access to treatment that was previously problematic for patients and clinicians.

### SUMMARY OF THE INVENTION

The compounds of the invention are amide prodrug conjugates that provide the therapeutic benefit of mexiletine but with reduced or eliminated GI side effects such as emesis.

The present invention is directed to mexiletine and p-OH mexiletine prodrugs of Formula I

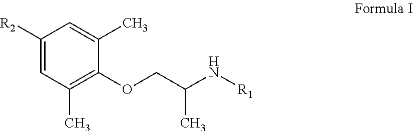

or a pharmaceutically acceptable salt thereof,

wherein,

R₁ is selected from hydrogen and

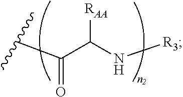

R₂ is selected from hydrogen,

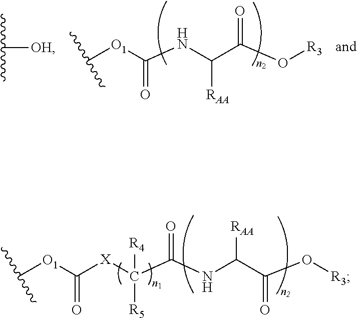

is the phenolic oxygen present in the unbound form of p-OH mexiletine;

X is (—NH—), or (—O—), or absent;

each occurrence of R_{AA} is independently a natural or non-natural amino acid side chain;

n₁ is an integer selected from 0 to 16;

each occurrence of n₂ is independently an integer selected from 1 to 9;

each occurrence of R₃ is independently selected from hydrogen, and an optionally substituted alkyl group;

each occurrence of R₄ and R₅ is independently selected from hydrogen,

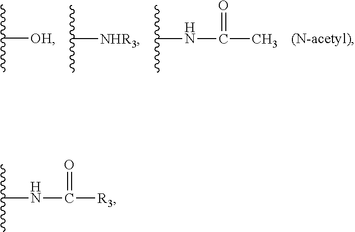

and an optionally substituted alkyl group;

wherein in the case of a double bond occurring in the carbon chain defined by n₁, R₄ is present and R₅ is absent on the carbons that form the double bond; and provided always that

at least R₁ is

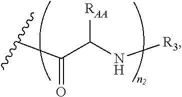

or alternatively, at least R₂ is either

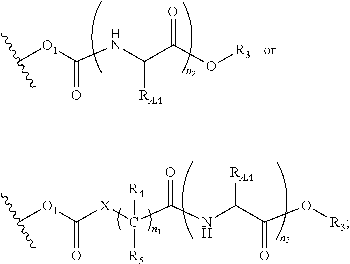

and provided that the compound is not one of the following compounds: HBr.glycine-(rac)mexiletine; AcOH.asparagine-(rac)mexiletine; TFA.tryptophan-(rac)mexiletine; HBr.alanine-(rac)mexiletine; AcOH-phenylalanine-(rac)mexiletine; and TFA.tryptophan-(rac)mexiletine. H₂O.

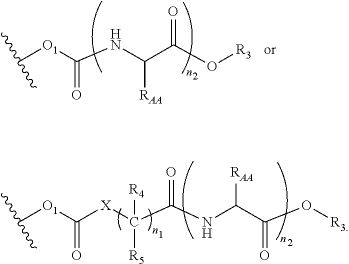

In one embodiment, R¹ is hydrogen and R² is

Preferably R² is

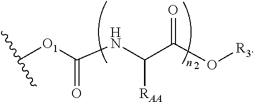

In one embodiment, R² is hydrogen and R¹ is

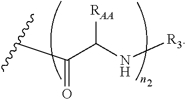

In an embodiment in which R² is

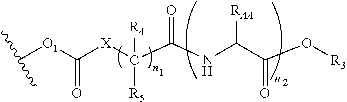

X is absent and n₁ is 1, 2 or 3. The value of n₂ in relation to R¹ is independent of its value in relation to R² when R² is other than hydrogen or hydroxy. In an embodiment, in the case of R¹, the value of n₂ is preferably 1, 2, 3 or 4; and more preferably n₂ is 1 or 2. Most preferably n₂ is 1.

In an embodiment, in the case of R², the value of n₂ is preferably 1, 2, 3 or 4. More preferably n₂ is 1 or 2, and most preferably n₂ is 1.

Amino acid residues used in accordance with the invention include: lysine, glycine, homoarginine, glutamic acid, methyl methionine and glutamine.

In one dicarboxylic acid linker embodiment, n₁ is 0, 1, 2, 3 or 4.

In one embodiment, each occurrence of n₂ is selected from 1, 2, 3, 4 and 5.

In a preferred embodiment, each occurrence of R³ is hydrogen. In an alternative embodiment, each occurrence of R³ is independently selected from an unsubstituted C₁₋₆ alkyl.

In one embodiment, each occurrence of R³ is independently selected from hydrogen, or an optionally substituted C₁₋₁₀ alkyl group. Preferably, the alkyl group, when present, is a C₁₋₆ alkyl group and, in some embodiments, does not include ^{t}Bu.

The compound of the invention is a compound of Formula (I) as defined with the proviso that the compound is not one of the excluded compounds. However, the exclusion does not always apply to the uses of the compounds and, in one embodiment of the uses of these compounds, the proviso does not apply. The abbreviation (rac) as used throughout the description refers to a racemic mixture. For example, in the case of (rac) mexiletine, this refers to DL mexiletine.

As mentioned above, the compound of Formula (I) is also not one of the following compounds: HBr.glycine-(rac)mexiletine; AcOH.asparagine-(rac)mexiletine; TFA.tryptophan-(rac)mexiletine; HBr.alanine-(rac)mexiletine; AcOH-phenylalanine-(rac)mexiletine; TFA.tryptophan-(rac)mexiletine.H₂O. The amino acids are in their natural configuration in this embodiment.

In one embodiment, there is a double bond in the chain defined by n₁. In this case, n₁ is from 2 to 16 and the C═C double bond is present in this portion. Alternatively, where n₁ is 1, the double bond can be present when X is N. In either case, the carbon atom or atoms engaged in the double bond bear only an R⁴ substituent and no R⁵ substituent. In an embodiment, no double bond is present irrespective of the value of n₁.

In another embodiment, the compound of Formula (I) is not one of the following compounds:
alanine-(rac)mexiletine amide;
β-methoxy aspartic acid-(rac)mexiletine amide;
α-methoxy aspartic acid-(rac)mexiletine amide;
asparagine-(rac)mexiletine amide;
glycine-(rac)mexiletine amide;
leucine-(rac)mexiletine amide;
methionine-(rac)mexiletine amide;
(rac)methionine-(rac)mexiletine amide;
phenylalanine-(rac)mexiletine amide;
alanine glycine glycine-(rac)mexiletine amide; or
tryptophan-(rac)mexiletine.

Preferably R4 is hydrogen or C1-4 alkyl at each occurrence, and more preferably each R⁴ is hydrogen.

Preferably R⁵ is hydrogen or C₁₋₄ alkyl at each occurrence, and more preferably each R⁵ is hydrogen.

In a preferred embodiment, the compound of the present invention has one prodrug moiety, and the prodrug moiety has one, two or three amino acids (i.e., n₂ is 1, 2 or 3), while R₃ is H.

There may be 1, 2, 3 or 4 independently selected optional substituents at any occurrence of such optional substitution. More usually, when present, there is 0 or 1 occurrences of optional substitution at each possibility of such substitution.

Where present and chemically compatible with the molecule, optional substituents are independently selected from the group comprising: hydroxy, C₁₋₄ alkyl, C₁₋₄ alkoxy, C₁₋₄ haloalkoxy, phenyl, benzyl, halogen, cyano, nitro, amino, amide and thio.

In another embodiment, at least one occurrence of n₂ is 1. In another embodiment, each occurrence of n₂ is 1. In yet another embodiment, at least one occurrence of n₂ is 2. In another embodiment, each occurrence of n₂ is 2.

In yet another embodiment, each occurrence of n₂ is 1 or 2 and each occurrence of R_{AA} is independently a natural amino acid side chain.

In one embodiment, the prodrug of the present invention has one prodrug moiety, and is a homopolymer of arginine or lysine, or a heteropolymer of arginine and lysine. In a further embodiment, there are 2, 3, 4, 5, 6 or 7 amino acids in the homopolymer or the heteropolymer (i.e., n₂ is 2, 3, 4, 5, 6 or 7).

In an embodiment, each occurrence of R^{AA} is independently an amino acid side chain i.e. an amino acid residue containing from 1 to 20 carbon atoms, or the residue is hydrogen (in the case of glycine). In the context of this invention, the term “amino acid” includes both natural amino acids (in their natural or non-natural stereochemical configuration) and synthetic amino acids. A natural amino acid is one of the twenty amino acids used for protein biosynthesis. The term may also include in some embodiments other amino acids which can be incorporated into proteins during translation (including pyrrolysine, ornathine and selenocysteine). Preferably, the or each R^{AA} is a naturally occurring amino acid.

Compositions of the mexiletine prodrug of the present invention are also provided herein. The compositions comprise at least one prodrug of the present invention (e.g., a prodrug of Formula I), or pharmaceutically acceptable salt thereof, and at least one pharmaceutically acceptable excipient.

In another aspect, the present invention provides a mexiletine conjugate of Formula (I) for use in the treatment of pain, such as neuropathic pain.

One embodiment of the present invention is a method of treating a disorder in a subject in need thereof with mexiletine. The method comprises orally administering a mexiletine prodrug of the present invention, a pharmaceutically acceptable salt thereof, or composition thereof, to a subject or group of subjects in need thereof, wherein the mexiletine prodrug is comprised of mexiletine or a mexiletine metabolite (e.g., para-OH (p-OH), meta-OH (m-OH) mexiletine or hydroxymethylmexiletine) covalently bonded to at least one amino acid or peptide of 2-9 amino acids in length. The mexiletine prodrug has one or two prodrug moieties. One prodrug moiety can be bound to the amino group of mexiletine or a mexiletine metabolite through a peptide bond. Alternatively or in addition, the mexiletine metabolite (e.g., p-OH, m-OH mexiletine or hydroxymethylmexiletine) can have a prodrug moiety bound to it through a carbamate or dicarboxylic acid linker at the phenolic oxygen. The amount of the mexiletine prodrug is preferably a therapeutically effective amount (e.g., an analgesic effective amount). The disorder may be one treatable with mexiletine. For example, the disorder may be neuropathic pain or arrhythmia.

In another embodiment, a prodrug of the present invention confers the benefit of reduced adverse gastrointestinal side effects (such as nausea and vomiting), compared to the parent compound, while at the same time improving upon the rate and consistency of achievement of therapeutic plasma drug concentrations.

Accordingly, in one embodiment, the present invention is directed to a method for minimizing the gastrointestinal side effects normally associated with administration of mexiletine. The method comprises orally administering a mexiletine prodrug of the present invention, pharmaceutically acceptable salt thereof, or composition thereof, to a subject in need thereof, wherein the mexiletine prodrug is comprised of mexiletine or a mexiletine metabolite (e.g., p-OH, m-OH mexiletine or hydroxymethylmexiletine) covalently bonded to at least one amino acid or peptide of 2-9 amino acids in length, and wherein upon oral administration, the prodrug or pharmaceutically acceptable salt minimizes, if not completely avoids, the gastrointestinal side effects usually seen after oral administration of the unbound mexiletine. The mexiletine prodrug has one or two prodrug moieties. One prodrug moiety can be bound to the amino group of mexiletine or a mexiletine metabolite through a peptide bond. Alternatively or in addition, the mexiletine metabolite (e.g., p-OH, m-OH mexiletine or hydroxymethylmexiletine) can have a prodrug moiety bound to it through a carbamate or dicarboxylic acid linker at the phenolic oxygen. The amount of the mexiletine is preferably a therapeutically effective amount (e.g., an analgesic effective amount).

In a further embodiment, the GI side effect associated with administration of mexiletine is selected from, but is not limited to, emesis, nausea ditherer and abdominal discomfort.

Another embodiment of the invention is directed to reducing the inter- and intra-subject variability of mexiletine serum levels. This will normally be during the treatment of pain or arrhythmias. The method comprises orally administering a mexiletine prodrug of the present invention, a pharmaceutically acceptable salt thereof, or composition thereof, to a subject or group of subjects in need thereof, wherein the mexiletine prodrug is comprised of mexiletine or a mexiletine metabolite (e.g., p-OH, m-OH mexiletine or hydroxymethylmexiletine) covalently bonded to at least one amino acid or peptide of 2-9 amino acids in length. The mexiletine prodrug has one or two prodrug moieties. One prodrug moiety can be bound to the amino group of mexiletine or a mexiletine metabolite through a peptide bond. Alternatively or in addition, the mexiletine metabolite (e.g., p-OH, m-OH mexiletine or hydroxymethylmexiletine) can have a prodrug moiety bound to it through a carbamate or dicarboxylic acid linker at the phenolic oxygen. The amount of the mexiletine prodrug is preferably a therapeutically effective amount (e.g., an analgesic effective amount).

Yet another embodiment of the invention related to improving the reproducibility of the bioavailability of mexiletine, in a subject in need thereof. The method comprises orally administering a mexiletine prodrug of the present invention, a pharmaceutically acceptable salt thereof, or composition thereof, to a subject or group of subjects in need thereof, wherein the mexiletine prodrug is comprised of mexiletine or a mexiletine metabolite (e.g., p-OH, m-OH mexiletine or hydroxymethylmexiletine) covalently bonded to at least one amino acid or peptide of 2-9 amino acids in length. The mexiletine prodrug has one or two prodrug moieties. One prodrug moiety can be bound to the amino group of mexiletine or a mexiletine metabolite through a peptide bond. Alternatively or in addition, the mexiletine metabolite (e.g., p-OH, m-OH mexiletine or hydroxymethylmexiletine) can have a prodrug moiety bound to it through a carbamate or dicarboxylic acid linker at the phenolic oxygen. The amount of the mexiletine prodrug is preferably a therapeutically effective amount (e.g., an analgesic effective amount).

Thus, the present invention relates to natural and/or non-natural amino acids and short-chain peptide prodrugs of mexiletine and its active metabolites. Without wishing to be bound to any particular theory, the prodrug portion of the compound (i.e., the amino acid and/or peptide portion) serves to temporarily protect the gut from the local actions of the drug or its active metabolite, while still delivering a pharmacologically effective amount of the drug/metabolite for the reduction or elimination of neuropathic pain, or treatment of arrhythmia. Such temporary inactivation reduces the profound and highly undesirable emetic side-effects of this drug. The prodrugs of the present invention also provide a means of not only accelerating that rate of attainment of maximum plasma concentrations—and hence onset of pain relief—but also improving the reproducibility of bioavailability of the drug ensuring a more consistent patient response both within and between patients. These conferred attributes serve to ensure improved analgesic efficacy and better patient compliance.

The present invention is also concerned with a group of compounds that enjoy the same or better activity than mexiletine itself. In this group of compounds, the emetic effect may still occur but the therapeutic activity is equal to, or more usually better than mexiletine. These compounds will be expected to be similar to mexiletine itself or more potent than mexiletine in the treatment of pain and arrhythmias.

Thus, according to another aspect of the present invention, there is provided a compound of Formula (IA)

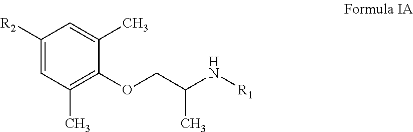

wherein R₁ is

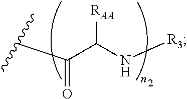

R² is selected from H and —OH;

each occurrence of R_{AA} is independently a natural or non-natural amino acid side chain containing from 1 to 20 carbon atoms or hydrogen;

n₂ is an integer selected from 1, 2, or 3;

R³ is selected from hydrogen and an optionally substituted C₁-4 alkyl group; provided that

the compound is not one of the following compounds:_HBr.glycine-(rac)mexiletine; AcOH.asparagine-(rac)mexiletine; TFA.tryptophan-(rac)mexiletine; HBr.alanine-(rac)mexiletine; AcOH-phenylalanine-(rac)mexiletine; or TFA.tryptophan-(rac)mexiletine.H₂O. In the above exclusion, the amino acids are in their natural configuration.

In another embodiment, the compound of Formula (IA) is not one of the following compounds:
alanine-(rac)mexiletine amide;
β-methoxy aspartic acid-(rac)mexiletine amide;
α-methoxy aspartic acid-(rac)mexiletine amide;
asparagine-(rac)mexiletine amide;
glycine-(rac)mexiletine amide;
leucine-(rac)mexiletine amide;
methionine-(rac)mexiletine amide;
methionine-(rac)mexiletine amide;
phenylalanine-(rac)mexiletine amide; or
alanine glycine glycine-(rac)mexiletine amide; or
tryptophan(rac)mexiletine.

In another embodiment, only on the compound of Formula (IA), R_{AA} is a natural amino acid side chain.

In an embodiment, the value of n₂ is preferably 1 or 2, and most preferably n₂ is 1.

In a preferred embodiment, R³ is hydrogen. In an alternative embodiment, R³ is an unsubstituted C₁₋₆ alkyl.

The compound of the invention is a compound of Formula (IA) as defined with the proviso that the compound is not one of the excluded compounds. However, the exclusion does not always apply to the uses of the compounds and, in one embodiment of the uses of these compounds, the proviso does not apply.

In another embodiment, the compound of Formula (IA) is selected from the group comprising:
mexiletine pipecolic acid amide; and
mexiletine dimethyl glycine amide.

These and other embodiments of the invention are disclosed or are apparent from and encompassed by the following Detailed Description.

### BRIEF DESCRIPTION OF THE DRAWINGS

FIG. 1 is a graph showing a voltage protocol for hNav1.x test procedure.

FIG. 2 is a graph showing the effects of (1) mexiletine, (2) mexiletine lysine amide, and (3) mexiletine glycine amide on electrical field stimulated contractions of isolated rabbit stomach circular smooth muscle preparation.

### DETAILED DESCRIPTION OF THE INVENTION

### Definitions

As used herein:

The term “peptide” refers to an amino acid chain consisting of 2 to 9 amino acids, unless otherwise specified. In preferred embodiments, the peptide used in the present invention is 2 or 3 amino acids in length. In one embodiment, a peptide can be a branched peptide. In this embodiment, at least one amino acid side chain in the peptide is bound to another amino acid (either through one of the termini or the side chain).

The term “amino acid” refers both to naturally occurring and non-naturally occurring amino acids. The amino acids contemplated for use in the prodrugs of the present invention include both natural and non-natural amino acids, preferably natural amino acids. The side chains R_{AA} can be in either the (R) or the (S) configuration. Additionally, both D and L amino acids are contemplated for use in the present invention.

A “natural amino acid” is one of the twenty amino acids used for protein biosynthesis as well as other amino acids which can be incorporated into proteins during translation (including pyrrolysine and selenocysteine). A natural amino acid generally has the formula

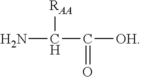

R_{AA} is referred to as the amino acid side chain, or in the case of a natural amino acid, as the natural amino acid side chain. The natural amino acids include glycine, alanine, valine, leucine, isoleucine, aspartic acid, glutamic acid, serine, threonine, glutamine, asparagine, arginine, lysine, proline, phenylalanine, tyrosine, tryptophan, cysteine, methionine and histidine (see Table 1).

In one embodiment, an amino acid side chain is bound to another amino acid. In a further embodiment, side chain is bound to the amino acid via the amino acid's N-terminus, C-terminus, or side chain.

Examples of natural amino acid sidechains include hydrogen (glycine), methyl (alanine), isopropyl (valine), sec-butyl (isoleucine), —CH₂CH(CH₃)₂ (leucine), benzyl (phenylalanine), p-hydroxybenzyl (tyrosine), —CH₂OH (serine), —CH(OH)CH₃ (threonine), CH₂-3-indoyl (tryptophan), —CH₂COOH (aspartic acid), —CH₂CH₂COOH (glutamic acid), —CH₂C(O)NH₂ (asparagine), —CH₂CH₂C(O)NH₂ (glutamine), —CH₂SH, (cysteine), —CH₂CH₂SCH₃ (methionine), —(CH₂)₄NH₂ (lysine), —(CH₂)₃NHC(═NH)NH₂ (arginine) and —CH₂-3-imidazoyl (histidine).

TABLE 1
Natural Amino Acids (Used For Protein Biosynthesis)

| Amino acid | 3 letter code | 1-letter code |
|---|---|---|
| Alanine | ALA | A |
| Cysteine | CYS | C |
| Aspartic Acid | ASP | D |
| Glutamic Acid | GLU | E |
| Phenylalanine | PHE | F |
| Glycine | GLY | G |
| Histidine | HIS | H |
| Isoleucine | ILE | I |
| Lysine | LYS | K |
| Leucine | LEU | L |
| Methionine | MET | M |
| Asparagine | ASN | N |
| Proline | PRO | P |
| Glutamine | GLN | Q |
| Arginine | ARG | R |
| Serine | SER | S |
| Threonine | THR | T |
| Valine | VAL | V |
| Tryptophan | TRP | W |
| Tyrosine | TYR | Y |

A “non-natural amino acid” is an organic compound that is not among those encoded by the standard genetic code, or incorporated into proteins during translation. Non-natural amino acids, thus, include amino acids or analogs of amino acids other than the 20 naturally-occurring amino acids used for protein biosynthesis and include, but are not limited to, the D-isostereomers of amino acids.

Examples of non-natural amino acids include, but are not limited to: citrulline, homocitrulline, hydroxyproline, homoarginine, homoserine, homotyrosine, homoproline, ornithine, 4-amino-phenylalanine, sarcosine, biphenylalanine, homophenylalanine, 4-amino-phenylalanine, 4-nitro-phenylalanine, 4-fluoro-phenylalanine, 2,3,4,5,6-pentafluoro-phenylalanine, para-amino benzoic acid, norleucine, cyclohexylalanine, α-aminoisobutyric acid, N-methyl-alanine, N-methyl-glycine, N-methyl-glutamic acid, tert-butylglycine, α-aminobutyric acid, α-aminoisobutyric acid, 2-aminoisobutyric acid, 2-aminoindane-2-carboxylic acid, selenomethionine, lanthionine, dehydroalanine, γ-amino butyric acid, naphthylalanine, aminohexanoic acid, phenylglycine, pipecolic acid, 2,3-diaminoproprionic acid, tetrahydroisoquinoline-3-carboxylic acid, tert-leucine, tert-butylalanine, cyclohexylglycine, diethylglycine, dipropylglycine and derivatives thereof wherein the amine nitrogen has been mono- or di-alkylated.

The invention thus also envisages amino acid derivatives such as those mentioned above which have been functionalised by simple synthetic transformations known in the art) e.g. as described in “Protective Groups in Organic Synthesis” by TW Greene and PGM Wuts, John Wiley & Sons Inc (1999), and references therein.

The term “polar amino acid” refers to a hydrophilic amino acid having a side chain that is uncharged at physiological pH, but which has at least one bond in which the pair of electrons shared in common by two atoms is held more closely by one of the atoms. Genetically encoded polar amino acids include Asn (N), Gln (Q) Ser (S) and Thr (T).

The term “nonpolar amino acid” refers to a hydrophobic amino acid having a side chain that is uncharged at physiological pH and which has bonds in which the pair of electrons shared in common by two atoms is generally held equally by each of the two atoms (i.e., the side chain is not polar). Genetically encoded nonpolar amino acids include Leu (L), Val (V), Ile (I), Met (M), Gly (G) and Ala (A).

The term “aliphatic amino acid” refers to a hydrophobic amino acid having an aliphatic hydrocarbon side chain. Genetically encoded aliphatic amino acids include Ala (A), Val (V), Leu (L) and Ile (I).

The term “amino” refers to a —NH₂ group.

The term “alkyl,” as a group, refers to a straight or branched hydrocarbon chain containing the specified number of carbon atoms. When the term “alkyl” is used without reference to a number of carbon atoms, it is to be understood to refer to a C₁-C₁₀ alkyl. For example, C₁₋₁₀ alkyl means a straight or branched alkyl containing at least 1, and at most 10, carbon atoms. Preferred alkyl groups often include C₁₋₆ or C₁₋₄ alkyl groups. Examples of “alkyl” as used herein include, but are not limited to, methyl, ethyl, n-propyl, n-butyl, n-pentyl, isobutyl, isopropyl, t-butyl, hexyl, heptyl, octyl, nonyl and decyl.

“The term “substituted alkyl” or “optionally substituted alkyl” as used herein denotes alkyl radicals wherein at least one hydrogen is replaced by one more substituents such as, but not limited to, hydroxy, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition pertains whether the term is applied to a substituent itself or to a substituent of a substituent.

The term “heterocycle” refers to a stable 3- to 15-membered ring radical which consists of carbon atoms and from one to five heteroatoms independently selected from nitrogen, phosphorus, oxygen and sulphur at each occurrence.

The term “cycloalkyl” group as used herein refers to a non-aromatic monocyclic hydrocarbon ring of 3 to 8 carbon atoms such as, for example, cyclopropyl, cyclobutyl, cyclopentyl, cyclohexyl or cycloheptyl.

The term “substituted cycloalkyl” as used herein denotes a cycloalkyl group further bearing one or more substituents as set forth herein, such as, but not limited to, hydroxy, alkoxy, aryl (for example, phenyl), heterocycle, halogen, trifluoromethyl, pentafluoroethyl, cyano, cyanomethyl, nitro, amino, amide (e.g., —C(O)NH—R where R is an alkyl such as methyl), amidine, amido (e.g., —NHC(O)—R where R is an alkyl such as methyl), carboxamide, carbamate, carbonate, ester, alkoxyester (e.g., —C(O)O—R where R is an alkyl such as methyl) and acyloxyester (e.g., —OC(O)—R where R is an alkyl such as methyl). The definition pertains whether the term is applied to a substituent itself or to a substituent of a substituent.

The terms “keto” and “oxo” are synonymous, and refer to the group ═O.

The term “carbonyl” refers to a group —C(═O).

The term “carboxyl” refers to a group —CO₂H and consists of a carbonyl and a hydroxyl group (More specifically, C(═O)OH).

“Amide,” as used herein, refers to the group

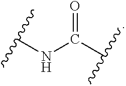

In the present invention, a prodrug moiety can be bonded to mexiletine via an amide linkage. In this embodiment, —N— is the amino nitrogen in the unbound mexiletine or mexiletine metabolite. Am amide linkage can be formed by reacting an amine with a carboxylic acid. This is the reaction that forms a peptide bond.

The terms “carbamate group,” and “carbamate,” concerns the group

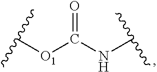

wherein the —O₁— is the phenolic oxygen in the unbound p-OH mexiletine molecule. Prodrug moieties described herein may be referred to based on their amino acid or peptide and the carbamate linkage. The amino acid or peptide in such a reference should be assumed to be bound via an amino terminus on the amino acid or peptide to the carbonyl linker and the phenolic oxygen of p-OH mexiletine, unless otherwise specified.

For example, val carbamate (valine carbamate) would have the formula

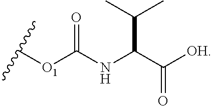

For a peptide, such as tyr-val carbamate, it should be assumed unless otherwise specified that the leftmost amino acid in the peptide is at the amino terminus of the peptide, and is bound via the carbonyl linker to p-OH mexiletine to form the carbamate prodrug.

The terms “dicarboxylic acid linker” and “dicarboxyl linker,” for the purposes of the present invention, are synonymous. The dicarboxylic acid linker refers to the group between mexiletine and the amino acid/peptide moiety:

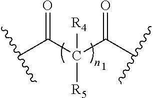

(—(CO)-(CR₄R₅)ₙ₁—(CO)—). Alternatively, the “dicarboxylic acid linker” can have the formula:

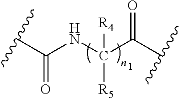

(—(CO)—(NH)—(CR₄R₅)ₙ₁—(CO)—), or the formula:

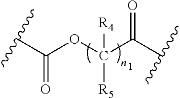

(—(CO)—(O)—(CR₄R₅)ₙ₁—(CO)—).

Regarding the dicarboxylic acid linker, one carbonyl group is bound to an oxygen atom in p-OH mexiletine, while the second carbonyl is bound to the N terminus of a peptide or amino acid, or an amino group of an amino acid side chain.

Dicarboxylic acid prodrug moieties described herein may be referred to based on their amino acid or peptide and the dicarboxyl linkage. The amino acid or peptide in such a reference should be assumed to be bound via an amino terminus on the amino acid or peptide to one carbonyl (originally part of a carboxyl group) of the dicarboxyl linker while the other is attached to p-OH mexiletine, unless otherwise specified. The dicarboxyl linker may or may not be variously substituted as stipulated earlier.

A non-limiting list of dicarboxylic acids for use with the present invention is given in Table 2. Although the dicarboxylic acids listed in Table 2 contain from 2 to 18 carbons, longer chain dicarboxylic acids can be used as linkers in the present invention. Additionally, the dicarboxylic acid linker can be substituted at one or more positions. A dicarboxylic acid, suitably activated, can be combined with an activated amino acid or peptide, and then reacted with p-OH mexiletine, to form a prodrug of the present invention. Prodrug syntheses procedures are discussed in more detail in the example section.

TABLE 2
Examples of Dicarboxylic Acids For Use With The Present Invention

| Common Name | IUPAC Name | Chemical Formula |
|---|---|---|
| Oxalic Acid | Ethanedioic Acid | HOOC—COOH |
| Malonic Acid | Propanedioic Acid | HOOC—(CH₂)—COOH |
| Succinic Acid | Butanedioic Acid | HOOC—(CH₂)₂—COOH |
| Glutaric Acid | Pentanedioic Acid | HOOC—(CH₂)₃—COOH |
| Adipic Acid | Hexanedioic Acid | HOOC—(CH₂)₄—COOH |
| Pimelic Acid | Heptanedioic Acid | HOOC—(CH₂)₅—COOH |
| Suberic Acid | Octanedioic Acid | HOOC—(CH₂)₆—COOH |
| Azelaic Acid | Nonanedioic Acid | HOOC—(CH₂)₇—COOH |
| Sebacic Acid | Decanedioic Acid | HOOC—(CH₂)₈—COOH |
| Undecanedioic Acid | Undecanedioic Acid | HOOC—(CH₂)₉—COOH |
| Dodecanedioic Acid | Dodecanedioic Acid | HOOC—(CH₂)₁₀—COOH |
| Brassylic Acid | Tridecanedioic Acid | HOOC—(CH₂)₁₁—COOH |
| | 1,11-Undecanedicarboxylic Acid | |
| Tetradecanedioic Acid | 1,12-Dodecanedicarboxylic Acid | HOOC—(CH₂)₁₂—COOH |
| Pentadecanedioic Acid | 1,15-Pentadecanedioic Acid | HOOC—(CH₂)₁₃—COOH |
| Thapsic Acid | Hexadecanedioic Acid | HOOC—(CH₂)₁₄—COOH |
| | Hexane-1,16-dioic Acid | |
| Heptadecanedioic Acid | 1,15-Pentadecanedicarboxylic Acid | HOOC—(CH₂)₁₅—COOH |
| Octadecanedioic Acid | 1,16-Tetradecanedicarboxylic Acid | HOOC—(CH₂)₁₆—COOH |

Dicarboxylic acid linkers of the present invention can have a nitrogen or oxygen atom bound to the first carbonyl group, i.e., X is (—NH—) or (—O—) in Formula 1, to give the linker structures

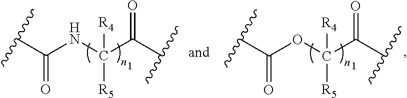

respectively. Examples of such dicarboxylic acid linkers are given in Table 2 and throughout the specification.

In one embodiment, the dicarboxylic acid linker is substituted. For example, one or more

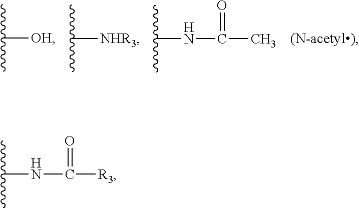

substituted alkyl groups, unsubstituted alkyl groups may be present (R₃, as defined by Formula 1). In these embodiments, X (—NH— or —O—, as defined by Formula 1) may be present or absent. Examples of dicarboxylic acid linkers are given in Table 2.

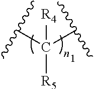

In one embodiment, the carbon chain in the dicarboxylic acid linker is unsaturated, and can have one or more double bonds. In these embodiments, n₁≧2 and R₅ is absent on the two carbons that form the double bond. One example of such a linker, fumaric acid, is given in Table 3.

TABLE 3
Dicarboxylic Acid Linkers For Use With The Present Invention

| Dicarboxylic Acid | | Valine Prodrug Moiety (p-OH |
|---|---|---|
| Linker Name | Structure | mexiletine phenolic oxygen shown) |
| N^{a}-Acetyl Aspartic Acid Linker | 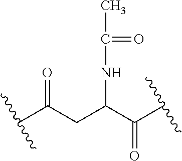 | 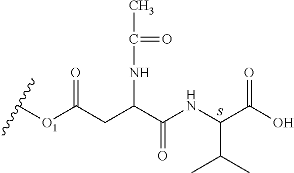 |
| N^{a}-Acetyl Glutamic Acid Linker | 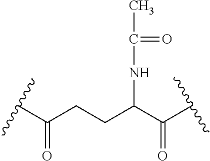 | 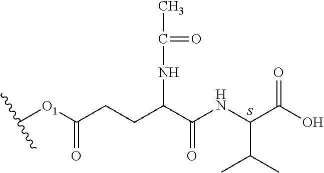 |
| Malic Acid Linker | 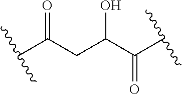 | 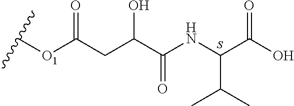 |
| Tartaric Acid Linker | 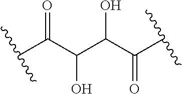 | 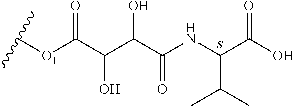 |
| Citramilic Acid Linker | 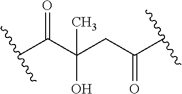 | 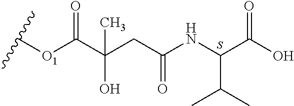 |
| β-Alanine Linker | 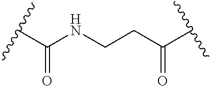 | 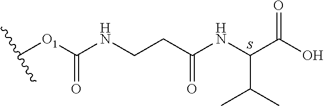 |
| γ-Aminobutyric Acid (GABA) Linker | 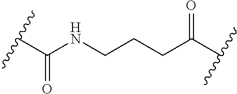 | 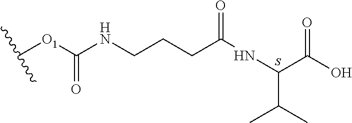 |
| 3-(Carboxyoxy) Butanoic Acid Linker | 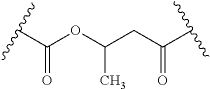 | 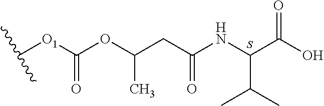 |
| 3-(Carboxyoxy) Propanoic Acid Linker | 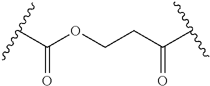 | 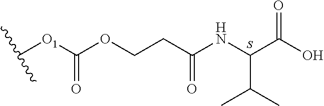 |
| 4-(Carboxyoxy) Butanoic Acid Linker | 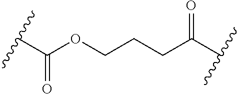 | 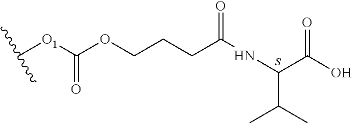 |
| Fumaric Acid Linker | 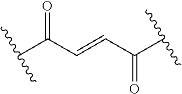 | 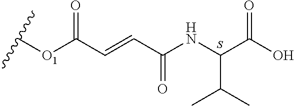 |

Examples of dicarboxylic acid prodrug moieties of the present invention include valine succinate, which has the formula

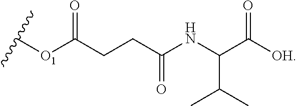

For a dipeptide, such as tyrosine-valine succinate, it should be assumed unless otherwise specified that the amino acid adjacent to the drug, in this case valine, is attached via the amino terminus to the dicarboxylic acid linker. The terminal carboxyl residue of the dipeptide (in this case tyrosine) forms the C (carboxyl) terminus.

The term “carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18^{th} Edition.

The phrase “pharmaceutically acceptable” refers to molecular entities and compositions that are generally regarded as safe. In particular, pharmaceutically acceptable carriers used in the practice of this invention are physiologically tolerable and do not typically produce an allergic or similar untoward reaction (for example, gastric upset, dizziness and the like) when administered to a patient. Preferably, as used herein, the term “pharmaceutically acceptable” means approved by a regulatory agency of the appropriate governmental agency or listed in the U.S. Pharmacopoeia or other generally recognized pharmacopoeia for use in animals, and more particularly in humans.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the present application includes both one and more than one such excipient.

The term “treating” includes: (1) preventing or delaying the appearance of clinical symptoms of the state, disorder or condition developing in an animal that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a patient to be treated is either statistically significant or at least perceptible to the patient or to the physician.

The term “subject” includes humans and other mammals, such as domestic animals (e.g., dogs and cats).

“Effective amount” means an amount of a prodrug or composition of the present invention sufficient to result in the desired therapeutic response. The therapeutic response can be any response that a user (e.g., a clinician) will recognize as an effective response to the therapy. The therapeutic response will generally be analgesia and/or an amelioration of one or more gastrointestinal side effect symptoms that are present when mexiletine or p-OH mexiletine in the prodrug is administered in its active form. It is further within the skill of one of ordinary skill in the art to determine appropriate treatment duration, appropriate doses, and any potential combination treatments, based upon an evaluation of therapeutic response.

The term “active ingredient,” unless specifically indicated, is to be understood as referring to mexiletine or a mexiletine metabolite portion of the prodrug, for example, the p-OH mexiletine portion of a prodrug of the present invention, as described herein.

“Mexiletine metabolite,” as used herein, refers to p-OH mexiletine

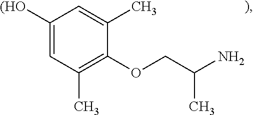

m-OH mexiletine

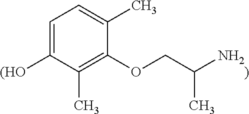

or hydroxymethylmexiletine

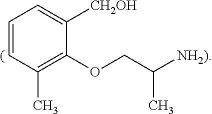

The term “salts” can include acid addition salts or addition salts of free bases. Suitable pharmaceutically acceptable salts (for example, of the carboxyl terminus of the amino acid or peptide) include, but are not limited to, metal salts such as sodium potassium and cesium salts; alkaline earth metal salts such as calcium and magnesium salts; organic amine salts such as triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. Pharmaceutically acceptable salts (of basic nitrogen centers) include, but are not limited to inorganic acid salts such as the hydrochloride, hydrobromide, sulfate, phosphate; organic acid salts such as trifluoroacetate and maleate salts; sulfonates such as methanesulfonate, ethanesulfonate, benzenesulfonate, p-toluenesulfonate, camphor sulfonate and naphthalenesulfonate; and amino acid salts such as arginate, gluconate, galacturonate, alaninate, asparginate and glutamate salts (see, for example, Berge, et al. (1977). “Pharmaceutical Salts,” J. Pharma. Sci. 66, 1).

The term “bioavailability,” as used herein, generally means the rate and/or extent to which the mexiletine or mexiletine metabolite is absorbed from a drug product and becomes systemically available, and hence available at the site of action. See Code of Federal Regulations, Title 21, Part 320.1 (2003 ed.). For oral dosage forms, bioavailability relates to the processes by which the active ingredient is released from the oral dosage form and moves to the site of action. Bioavailability data for a particular formulation provides an estimate of the fraction of the administered dose that is absorbed into the systemic circulation. Thus, the term “oral bioavailability” refers to the fraction of a dose of mexiletine given orally that is absorbed into the systemic circulation after a single administration to a subject. A preferred method for determining the oral bioavailability is by dividing the AUC of the mexiletine given orally by the AUC of the same mexiletine dose given intravenously to the same subject, and expressing the ratio as a percent. Other methods for calculating oral bioavailability will be familiar to those skilled in the art, and are described in greater detail in Shargel and Yu, Applied Biopharmaceutics and Pharmacokinetics, 4th Edition, 1999, Appleton & Lange, Stamford, Conn., incorporated herein by reference in its entirety.

Compounds of the Invention

In one embodiment, the mexiletine prodrug of the present invention has a prodrug moiety attached to mexiletine's amino nitrogen via an amide linkage. Alternatively, or additionally (i.e., the mexiletine prodrug has two prodrug moieties), the prodrugs of the present invention are novel amino acid and peptide prodrugs of a mexiletine metabolite (e.g., p-OH, m-OH mexiletine or hydroxymethylmexiletine) linked via a carbamate or dicarboxylic acid linker group the hydroxyloxygen. Preferably, the prodrugs of the present invention comprise mexiletine or p-OH mexiletine attached to a single amino acid or short peptide from two to nine amino acids in length.

In a dicarboxylic acid prodrug embodiment, the hydroxyl oxygen of the mexiletine metabolite (e.g., p-OH, m-OH mexiletine or hydroxymethylmexiletine) can be esterified with a dicarboxylic acid such as, but not limited to, malonic, succinic, glutaric, adipic or other longer chain dicarboxylic acid, or substituted derivative thereof (for representative examples of dicarboxylic acid linkers, see Tables 2 and 3). The amino acid or peptide may then be attached to the remaining carboxyl group via the N-terminal nitrogen on the peptide/amino acid, or a nitrogen present in an amino acid side chain (e.g., a lysine side chain).

In a mexiletine amide prodrug embodiment, a prodrug moiety (i.e., amino acid or peptide) can be bonded to mexiletine or a mexiletine metabolite's amino group. An amide linkage can be formed by reacting mexiletine or the mexiletine metabolite's amino group with a carboxylic acid. In a further mexiletine amide prodrug embodiment, the prodrug is a p-OH mexiletine prodrug and has two prodrug moieties. The second prodrug moiety can be bonded to p-OH, m-OH mexiletine or hydroxymethylmexiletine's hydroxylic oxygen via a dicarboxylic acid linkage or a carbamate group.

Advantages of the Compounds of the Invention

Without wishing to be bound to any particular theory, it is believed that the amino acid or peptide portion of the mexiletine or mexiletine metabolite prodrugs provided herein selectively exploit the inherent di- and tripeptide transporter Pept1 within the digestive tract to effect absorption. Once absorbed, the prodrugs are subject to hydrolysis, releasing the active drug into the systemic circulation. It is believed that mexiletine is subsequently released from the amino acid or peptide prodrug by hepatic and extrahepatic hydrolases that are, in part, present in blood and or plasma.

Such assisted absorption of the prodrugs by Pept1 can provide greater consistency in analgesic response possibly as the result of more consistent, oral bioavailability. As a result of this more reproducible, oral bioavailability, the prodrugs of the present invention offer a significant reduction of inter- and intra-subject variability of mexiletine plasma and CNS concentrations and, hence, significantly less fluctuation in pain relief for a single patient, or among a patient population. Thus, patient compliance is likely to be further improved as the result of this greater predictability of analgesic response.

Any locally mediated emesis (i.e., from within the gut lumen) associated with the administration of mexiletine can be potentially reduced if mexiletine could be transiently inactivated until absorbed. This inactivation can preclude direct exposure of the drug to the lower oesophageal sphincter and stomach. An inactive prodrug of mexiletine that is only activated post absorption could be one way of eliminating emesis and other adverse GI effects. As an alternative approach, prodrugs of active mexiletine metabolites can be employed (e.g., p-OH, m-OH mexiletine or hydroxymethylmexiletine). Para-hydroxymexiletine has been reported to retain around 25% of the sodium channel inhibitory activity of the parent molecule and may therefore be a useful drug in its own right (De Bellis et al. (2006). Brit. J. Pharmacol. 149, 300-310). If the adverse GI side effects (e.g., emesis) associated with mexiletine could be satisfactorily overcome by transient inactivation, the resultant product could provide a valuable addition to the currently limited armamentarium of drugs for the treatment of neuropathic pain.

Additionally, single amino acids and peptides would not be expected to present a toxicity risk; and the amino acid or peptide would also likely transiently inactivate mexiletine or its active metabolites due the profound change in overall structure and conferred water solubility. Additionally, judiciously chosen peptide conjugates could offer the potential for protracted or sustained release by their partial hydrolysis by peptidases such as trypsin, within the gut lumen. For example, the introduction of a C-terminus poly-arginine or poly-lysine fragment to the drug either directly or indirectly (e.g., through another amino acid such as glycine) may result in partial hydrolysis in the gut lumen and hence control the rate of delivery of the resultant potentially absorbable di- or tripeptidomimetic compound for absorption. Such absorption is then likely to be effected by active transporters such as Pept1, which is specific for di- and tripeptides.

Prodrugs of Mexiletine and it p-Hydroxy Metabolite of the Present Invention

In one embodiment, the present invention is directed to mexiletine and p-OH mexiletine prodrugs of Formula I.

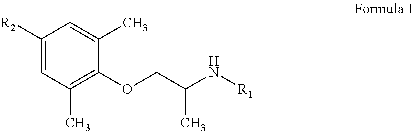

or a pharmaceutically acceptable salt thereof,

wherein,

R₁ is selected from hydrogen and

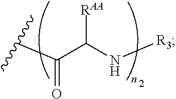

R₂ is selected from hydrogen,

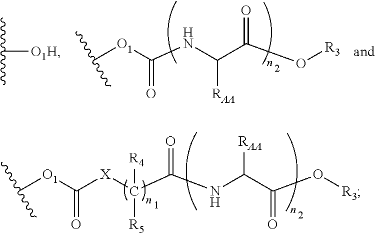

O₁ is the phenolic oxygen present in the unbound form of p-OH mexiletine;

X is (—NH—), (—O—), or absent;

Each occurrence of R_{AA} is independently a natural or non-natural amino acid side chain;

n₁ is an integer selected from 0 to 16;

Each occurrence of n₂ is independently an integer selected from 1 to 9;

Each occurrence of R₃ is independently selected from hydrogen, a substituted alkyl group or an unsubstituted alkyl group;

Each occurrence of R₄ and R₅ is independently selected from hydrogen,

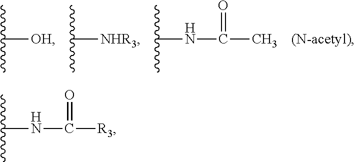

a substituted alkyl group and an unsubstituted alkyl group;

In the case of a double bond in the carbon chain defined by n₁, R₄ is present and R₅ is absent on the carbons that form the double bond; and

at least R₁ is

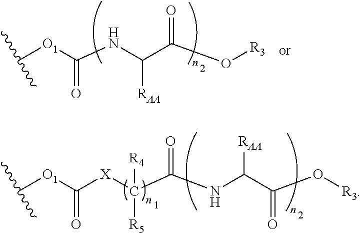

or alternatively, at least R₂ is

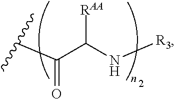

In one dicarboxylic acid linker embodiment, n₁ is 0, 1, 2, 3 or 4 and R₁ is H.

In another dicarboxylic acid linker embodiment, n₁ is 0, 1, 2, 3 or 4, R₁ is

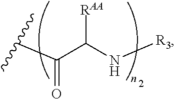

each occurrence of n₂ is 1, 2 or 3 and R₃ is H.

In one embodiment, each occurrence of n₂ is 1, 2, 3, 4 or 5.

In a preferred embodiment, the compound of the present invention has one prodrug moiety, and the prodrug moiety has one, two or three amino acids (i.e., n₂ is 1, 2 or 3), while R₃ is H.

In another embodiment, n₂ is 1. In yet another embodiment, n₂ is 2.

In yet another embodiment, each occurrence of n₂ is 1 or 2 and each occurrence of R_{AA} is independently a natural amino acid side chain.

In one embodiment, the prodrug has one prodrug moiety, and is a homopolymer of arginine or lysine, or a heteropolymer of arginine and lysine. In a further embodiment, there are 2, 3, 4, 5, 6 or 7 amino acids in the homopolymer or the heteropolymer (i.e., n₂ is 2, 3, 4, 5, 6 or 7).

In one dicarboxylic acid linker embodiment, n₁ is 0, 1, 2 or 3. In a further embodiment n₁ is 0, 1, 2 or 3 while each occurrence of R₃, R₄ and R₅ is hydrogen.

In one embodiment, each occurrence of n₂ is 1, 2, 3, 4 or 5. In a further embodiment, n₂ is 1, 2, 3, 4 or 5 while each occurrence of R₃ is hydrogen.

In a preferred embodiment, the compound of the present invention has one prodrug moiety, and the prodrug moiety has one, two or three amino acids (i.e., n₂ is 1, 2 or 3), while R₃ is H.

In another embodiment, n₂ is 1. In yet another embodiment, n₂ is 2.

In yet another embodiment, each occurrence of n₂ is 1 or 2 and each occurrence of R_{AA} is independently a natural amino acid side chain.

In one embodiment, the peptide prodrug moiety attached to the drug's amino function is a glycine or lysine residue. In a further embodiment, the peptide prodrug moiety of the present invention incorporates an arginine or lysine residue directly next to the glycine or lysine by a peptide bond between the glycine or lysine's C-terminus and the arginine or lysine's amino terminus (or side chain nitrogen). Therefore, the following dipeptides are contemplated by the present invention, either solely as dipeptide prodrug moieties, or alternatively, as portions of prodrug moieties—(1) Glycine-Arginine, (2) Glycine-Lysine, (3) Lysine-Arginine, (4) Lysine-Lysine. In these embodiments, the first amino acid listed is bound to the mexiletine.

In another embodiment the peptide prodrug moiety is attached to the p-hydroxy group in mexiletine's hydroxy metabolite. Preferred amino acid attached via a carbamate or dicarboxylic acid bridges would include, but not be restricted to, valine, leucine, isoleucine or methionine.

Peptides comprising any of the naturally occurring amino acids, as well as non-natural amino acids, can be used in the present invention. If non-natural amino acids are employed as a peptide prodrug moiety, or portion thereof, the peptide can include solely non-natural amino acids, or alternatively, a combination of natural and non-natural amino acids.

The amino acids employed in the prodrugs for use with the present invention are preferably in the L configuration. The present invention also contemplates prodrugs of the invention comprised of amino acids in the D configuration, or mixtures of amino acids in the D and L configurations.

In one embodiment, an amide-linked mexiletine prodrug of Formula II is provided. In another embodiment, an amide-linked p-OH mexiletine prodrug of Formula III is provided. For Formulae II-III, R₃, R_{AA} and n₂ are defined as provided for Formula I. Pharmaceutically acceptable salts of the prodrugs of Formulae II-III are also encompassed by the present invention.

In this embodiment, the prodrug of the present invention comprises mexiletine or p-OH mexiletine covalently attached to an amino acid or short peptide through an amide linkage, wherein the amide linkage formed from the amine function in the drug and carboxyl function of the amino acid (or C-terminus of peptide). As stated above, the amino acids used in the present invention may be natural or non-natural. For example, glycine, lysine, arginine, citrulline, ornithine, and can either be covalently attached to the mexiletine or p-OH mexiletine as a single amino acid, or as a portion of a peptide.

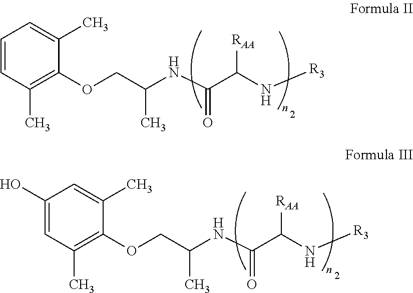

In one Formula II embodiment, n₂ is either 1, 2, 3 or 4 and R₃ is H. In another Formula II embodiment, n₂ is either 1, 2, 3 or 4 and R₃ is an alkyl group.

In one Formula III embodiment, n₂ is either 1, 2, 3 or 4 and R₃ is H. In another Formula III embodiment, n₂ is either 1, 2, 3 or 4 and R₃ is an alkyl group.

In another embodiment of Formula II and/or Formula III, n₂ is 1, 2, 3, 4 or 5. In a further embodiment, n₂ is 1, 2, 3, 4 or 5 while R₃ is hydrogen. In a preferred embodiment of Formula II or Formula III, the prodrug moiety has one, two or three amino acids (i.e., n₂ is 1, 2 or 3), while R₃ is H. In another embodiment, n₂ is 1. In yet another embodiment, n₂ is 2. In yet another embodiment, n₂ is 1 or 2 and each occurrence of R_{AA} is independently a natural amino acid side chain.

In another embodiment of the invention, p-OH mexiletine carbamate prodrugs of Formulae IV are provided. For Formula IV, O₁, R₃, R_{AA} and n₂ are defined as provided for Formula I.

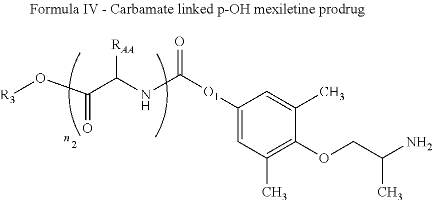

In one Formula IV embodiment, n₂ is either 1, 2, 3 or 4 and R₃ is H. In a further Formula IV embodiment, n₂ is either 1, 2, 3 or 4 and R₃ is H. In yet a further embodiment, each occurrence of R_{AA} is a natural amino acid side chain.

In another Formula IV embodiment, n₂ is 1, 2, 3, 4 or 5. In a further embodiment, n₂ is 1, 2, 3, 4 or 5 while R₃ is hydrogen. In a preferred Formula IV embodiment, the prodrug moiety has one, two or three amino acids (i.e., n₂ is 1, 2 or 3), while R₃ is H. In an alternative Formula IV embodiment, the prodrug moiety has one, two or three amino acids (i.e., n₂ is 1, 2 or 3), while R₃ is an alkyl group.

In yet another embodiment, n₂ is 1. In yet another embodiment, n₂ is 2. In yet another embodiment, n₂ is 1 or 2 and each occurrence of R_{AA} is independently a natural amino acid side chain. In another embodiment, at least one occurrence of R_{AA} is a non-natural amino acid side chain.

Another embodiment of the present invention is directed to dicarboxylic acid linked p-OH mexiletine prodrugs of Formula V. For Formula V, O₁, R₃, R₄, R₅, R_{AA}, —X—, n₁ and n₂ are defined as provided for Formula I.

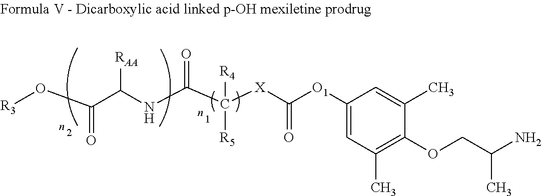

In one Formula V embodiment, n₁ is an integer selected from 0 to 4. In a further embodiment, R₃ is H and n₂ is 1, 2 or 3.

In one Formula V embodiment of Formula V, n₁ is 0, 1, 2 or 3. In a further embodiment, n₁ is 0, 1, 2 or 3 while each occurrence of R₃, R₄ and R₅ is hydrogen.

In yet a further embodiment, n₂ is 1, 2 or 3. In another Formula V embodiment, X is absent and n₁ is 1, 2 or 3. In even a further Formula V embodiment, X is absent, n₁ is 1, 2 or 3, n₂ is 1 or 2 and R₃, R₄ and R₅ are each hydrogen.

In one Formula V embodiment, X is —NH—, n₁ is 0, 1, 2 or 3, n₂ is 1, 2 or 3 and R₃, R₄ and R₅ are each H. In a further embodiment, n₁ is 2.

In one Formula V embodiment, X is —O—, n₁ is 0, 1, 2 or 3, n₂ is 1, 2 or 3 and R₃, R₄ and R₅ are each H. In a further embodiment, n₁ is 2.

In another Formula V embodiment, X is absent, n₁ is 1, 2 or 3 and n₂ is 1, 2 or 3. In yet another Formula V embodiment, X is absent and n₁ is 1 or 2 and n₂ is 1, 2, 3, 4 or 5.

In a preferred Formula V embodiment, the prodrug moiety of the present invention has one or two amino acids (i.e., n₂ is 1 or 2). In one embodiment, n₁ is 1 or 2 while n₂ is 1, 2 or 3.

In one Formula V embodiment, X is —O—, n₁ is 0, 1 or 2, n₂ is 1 or 2 and R₃ is H. In a further embodiment, at least one occurrence of R₄ is

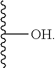

In one embodiment, X is —NH—, n₁ is 0, 1 or 2, n₂ is 1 or 2 and R₃ is H. In a further embodiment, at least one occurrence of R₄ is

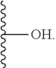

In a preferred Formula V embodiment, n₂ is 1, 2 or 3 while R₃, R₄ and R₅ are H. In another embodiment, n₂ is 1. In yet another embodiment, n₂ is 2. In yet another Formula V embodiment, n₂ is 1 or 2 and each occurrence of R_{AA} is independently a natural amino acid side chain.

In another embodiment of the present invention, the p-OH mexiletine prodrug of the present invention is a dicarboxylic acid linked prodrug of Formula VI or VII. For both Formula VI and Formula VII, O₁, R₃, R₄, R₅, R_{AA}, n₁ and n₂ are defined as provided for Formula I.

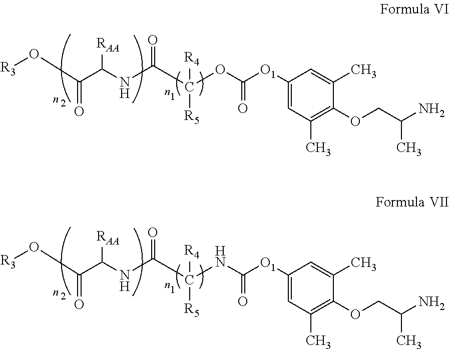

In one dicarboxylic acid linker embodiment of Formula VI and/or Formula VII, n₁ is 0, 1, 2 or 3. In a further embodiment, n₁ is 0, 1, 2 or 3 while each occurrence of R₃, R₄ and R₅ is hydrogen.

In yet a further embodiment of Formula VI and/or Formula VII, n₂ is 1, 2 or 3. In a further Formula VI and/or Formula VII embodiment, n₁ is 1, 2 or 3. In even a further embodiment, n₁ is 1, 2 or 3, n₂ is 1 or 2 and R₁, R₂ and R₃ are each hydrogen.

In one Formula VI and/or Formula VII embodiment, n₁ is 0, 1, 2 or 3, n₂ is 1, 2 or 3 and R₃, R₄ and R₅ are each H. In a further embodiment, n₁ is 2.

In a preferred Formula VI and/or Formula VII embodiment, the prodrug moiety of the present invention has one, two or three amino acids (i.e., n₂ is 1, 2 or 3). In one embodiment, n₁ is 1 or 2 while n₂ is 1, 2 or 3.

In one Formula VI and/or Formula VII embodiment, n₁ is 0, 1 or 2, n₂ is 1 or 2 and R₃ is H. In a further embodiment, at least one occurrence of R₄ is

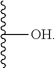

In one Formula VI and/or Formula VII embodiment, n₁ is 0, 1 or 2, n₂ is 1 or 2 and R₃ is H. In a further embodiment, at least one occurrence of R₄ is

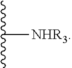

In a preferred Formula VI and/or Formula VII embodiment, n₂ is 1, 2 or 3 while R₃, R₄ and R₅ are H. In another embodiment, n₂ is 1. In yet another embodiment, n₂ is 2. In yet another Formula VI and/or Formula VII embodiment, n₂ is 1 or 2 and each occurrence of R_{AA} is independently a natural amino acid side chain.

In yet another embodiment of the invention, a p-OH mexiletine prodrug of Formula VIII, or a pharmaceutically acceptable salt thereof, is provided. For Formula VIII, O₁, R₃, R_{AA}, n₁ and n₂ are defined as provided for Formula I.

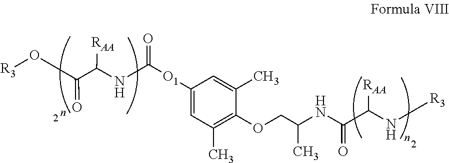

In one Formula VIII embodiment, at least one occurrence of n₂ is 1, 2, 3 or 4 and at least one occurrence of R₃ is H. In a further embodiment, each occurrence of n₂ is selected from 1, 2, 3 and 4, and each occurrence R₃ is H.

In another Formula VIII embodiment, at least one occurrence of n₂ is 1, 2, 3, 4 or 5. In a further embodiment, at least one occurrence of n₂ is 1, 2, 3, 4 or 5 while at least one occurrence of R₃ is hydrogen. In a further embodiment, each occurrence of n₂ is 1, 2, 3, 4 or 5 while each occurrence of R₃ is hydrogen. In yet another Formula VIII embodiment, each occurrence of n₂ is 1, 2, 3, 4 or 5 while at least one occurrence of R₃ is an alkyl group.

In a preferred embodiment of Formula VIII, at least one prodrug moiety has one, two or three amino acids (i.e., n₂ is 1, 2 or 3), while at least one occurrence of R₃ is H. In another Formula VIII embodiment, at least one occurrence of n₂ is 1. In yet another Formula VIII embodiment, at least one occurrence of n₂ is 2. In yet another Formula VIII embodiment, at least one occurrence of n₂ is 1 or 2 and each occurrence of R_{AA} is independently a natural amino acid side chain. In another Formula VIII embodiment, both occurrences of n₂ are selected from 1, 2 and 3.

Another embodiment of the present invention is directed to a p-OH mexiletine prodrug of Formula IX, or a pharmaceutically acceptable salt thereof. For Formula IX, O₁, X—, R₃, R₄, R₅, n₁ and n₂ are defined as provided for Formula I.

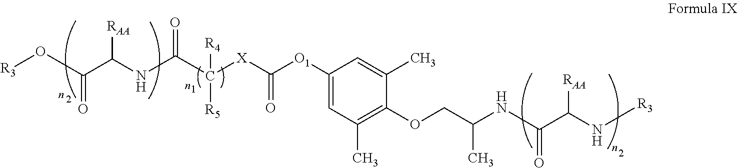

In one Formula IX embodiment, n₁ is an integer selected from 0 to 4.

In one dicarboxylic acid linker embodiment of Formula IX, n₁ is 0, 1, 2 or 3. In a further embodiment, n₁ is 0, 1, 2 or 3 while each occurrence of R₃, R₄ and R₅ is hydrogen.

In yet a further embodiment, n₂ is 1, 2 or 3. In another Formula IX embodiment, —X— is absent and n₁ is 1, 2 or 3. In even a further Formula IX embodiment, each occurrence of n₂ is 1 or 2 and R₃, R₄ and R₅ are each hydrogen.

In one Formula IX embodiment, —X— is —NH—, n₁ is 0, 1, 2 or 3, n₂ is 1, 2 or 3 and R₃, R₄ and R₅ are each H. In a further embodiment, n₁ is 2.

In one Formula IX embodiment, —X— is —O—, n₁ is 0, 1, 2 or 3, each occurrence of n₂ is 1, 2, 3, 4 or 5, and R₃, R₄ and R₅ are each H. In a further embodiment, n₁ is 2.

In another Formula IX embodiment, —X— is absent, n₁ is 1, 2 or 3 and n₂ is 1, 2 or 3. In yet another Formula IX embodiment, —X— is absent and n₁ is 1 or 2 and each occurrence of n₂ is 1, 2, 3, 4 or 5.

In a preferred Formula IX embodiment, the prodrug moiety of the present invention has one or two amino acids (i.e., n₂ is 1 or 2). In one embodiment, n₁ is 1 or 2 while each occurrence of n₂ is 1, 2 or 3.

In one Formula IX embodiment, —X— is —O—, n₁ is 0, 1 or 2, n₂ is 1 or 2 and R₃ is H. In a further embodiment, at least one occurrence of R₄ is

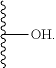

In another Formula IX embodiment, —X— is —NH—, n₁ is 0, 1 or 2, each occurrence of n₂ is 1 or 2, and R₃ is H. In a further embodiment, at least one occurrence of R₄ is

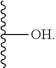

In one Formula IX embodiment, —X— is —O—, n₁ is 0, 1 or 2, n₂ is 1 or 2 and R₃ is H. In a further embodiment, at least one occurrence of R₄ is

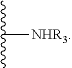

In another Formula IX embodiment, —X— is —NH—, n₁ is 0, 1 or 2, each occurrence of n₂ is 1 or 2 and R₃ is H. In a further embodiment, at least one occurrence of R₄ is

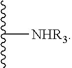

In a preferred Formula IX embodiment, n₂ is 1, 2 or 3 while R₁, R₂ and R₃ are H. In another embodiment, at least one occurrence of n₂ is 1. In yet another embodiment, n₂ is 2. In yet another Formula IX embodiment, n₂ is 1 or 2 and each occurrence of R_{AA} is independently a natural amino acid side chain.

Other embodiments of the present invention are directed to p-OH mexiletine prodrugs of Formulae X and XI, or pharmaceutically acceptable salts thereof. For Formulae X and XI, O₁, R₃, R₄, R₅, R_{AA}, n₁ and n₂ are defined as provided for Formula I.

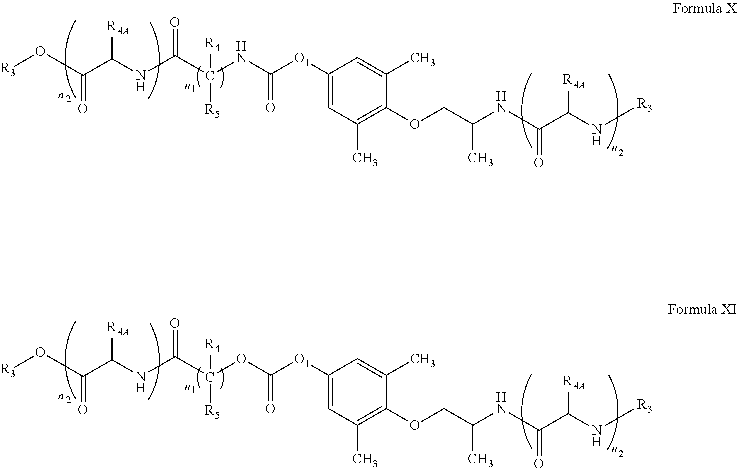

In one embodiment of Formula X and/or Formula XI, n₁ is 0, 1, 2 or 3. In a further embodiment, n₁ is 0, 1, 2 or 3 while each occurrence of R₃, R₄ and R₅ is hydrogen.

In yet a further embodiment of Formula X and/or Formula XI, n₂ is 1, 2 or 3. In another Formula X and/or Formula XI embodiment, n₁ is 1, 2 or 3. In even a further embodiment, n₁ is 1, 2 or 3, each occurrence of n₂ is 1, 2, 3 or 4, and R₃, R₄ and R₅ are each hydrogen.

In one Formula X and/or Formula XI embodiment, n₁ is 0, 1, 2 or 3, each occurrence of n₂ is 1, 2 or 3 and R₃, R₄ and R₅ are each H. In a further embodiment, n₁ is 2.

In a preferred Formula X and/or Formula XI embodiment, both prodrug moieties of the present invention have one, two, three or four amino acids (i.e., n₂ is 1, 2, 3 or 4). In one embodiment, n₁ is 1 or 2 while each occurrence of n₂ is 1, 2 or 3.

In one Formula X and/or Formula XI embodiment, n₁ is 0, 1 or 2, at least one occurrence of n₂ is 1 or 2 and R₃ is H. In a further embodiment, at least one occurrence of R₄ is

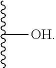

In one Formula X and/or Formula XI embodiment, n₁ is 0, 1 or 2, each occurrence of n₂ is 1, 2 or 3, and R₃ is H. In a further embodiment, at least one occurrence of R₄ is

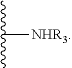

In a preferred Formula X and/or Formula XI embodiment, n₂ is 1, 2 or 3 while R₁, R₂ and R₃ are H. In another embodiment, at least one occurrence of n₂ is 1. In yet another embodiment, at least one occurrence of n₂ is 2. In yet another Formula X and/or Formula XI embodiment, each occurrence of n₂ is 1 or 2 and each occurrence of R_{AA} is independently a natural amino acid side chain.

Prodrugs of Meta-OH Mexiletine

Although the Formulae I-XI embodiments described above are directed to mexiletine and p-OH mexiletine prodrugs, the present disclosure also encompasses prodrugs of other mexiletine prodrugs. Accordingly, prodrugs of m-OH mexiletine and hydroxymethylmexiletine, are within the scope of this disclosure.

For example, in one embodiment, the present invention is directed to meta-OH mexiletine prodrugs, encompassed by Formula XII.

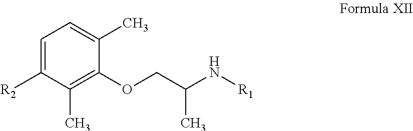

or a pharmaceutically acceptable salt thereof, wherein,

R₁ is selected from hydrogen and

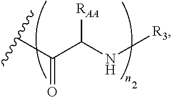

R₂ is selected from

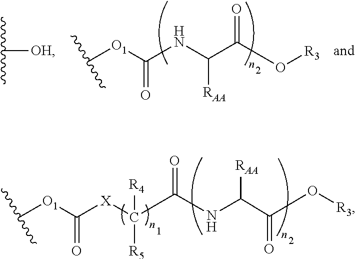

O₁ is the phenolic oxygen present in the unbound form of meta-OH mexiletine;

X is (—NH—), (—O—), or absent;

Each occurrence of R_{AA} is independently a natural or non-natural amino acid side chain;

n₁ is an integer selected from 0 to 16;

Each occurrence of n₂ is independently an integer selected from 1 to 9;

Each occurrence of R₃ is independently selected from hydrogen, a substituted alkyl group or an unsubstituted alkyl group;

Each occurrence of R₄ and R₅ is independently selected from hydrogen,

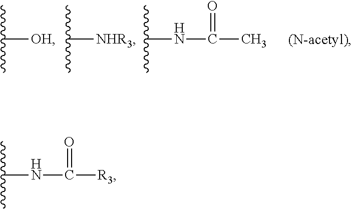

a substituted alkyl group and an unsubstituted alkyl group;

In the case of a double bond in the carbon chain defined by n₁, R₄ is present and R₅ is absent on the carbons that form the double bond; and

at least R₁ is

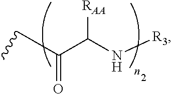

or alternatively, at least R₂ is

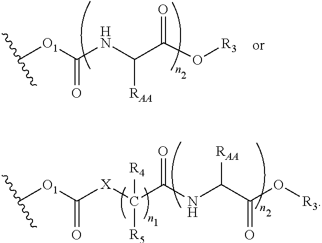

In one dicarboxylic acid linker embodiment, n₁ is 0, 1, 2, 3 or 4.

In one embodiment, each occurrence of n₂ is selected from 1, 2, 3, 4 and 5.

In a preferred embodiment, the compound of the present invention has one prodrug moiety, and the prodrug moiety has one, two or three amino acids (i.e., n₂ is 1, 2 or 3), while R₃ is H.

In another embodiment, n₂ is 1.

In yet another embodiment, n₂ is 2.

In yet another embodiment, each occurrence of n₂ is 1 or 2 and each occurrence of R_{AA} is independently a natural amino acid side chain.

In one embodiment, the prodrug moiety is a homopolymer of arginine or lysine, or a heteropolymer of arginine and lysine. In a further embodiment, there are 2, 3, 4, 5, 6 or 7 amino acids in the homopolymer or the heteropolymer (i.e., n₂ is 2, 3, 4, 5, 6 or 7).

As stated above, and provided in Formula XII, the para substituted prodrug moieties described herein (e.g., prodrugs of Formulae II-XI) can also be at the meta position. In these embodiments, the prodrug moiety at the para position is replaced with a hydrogen, while the prodrug moiety is attached at one of the carbon's adjacent to the para position. An example is shown below, for a carbamate linked meta-OH mexiletine prodrug (Formula XIII).

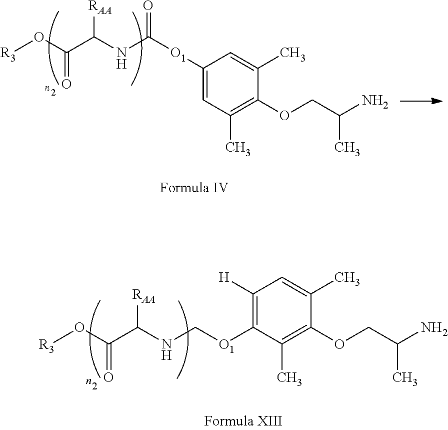

Prodrugs of Hydroxymethylmexiletine

In one embodiment, the present invention is directed to hydroxymethylmexiletine prodrugs, encompassed by Formula XIV.

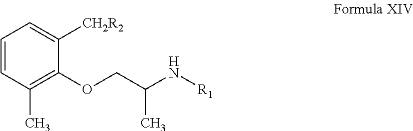

or a pharmaceutically acceptable salt thereof,

wherein,

R₁ is selected from hydrogen and

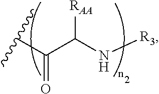

R₂ is selected from

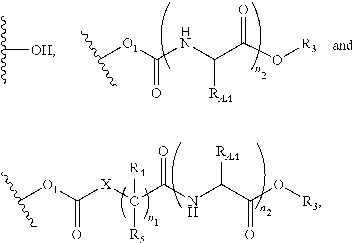

O₁ is the oxygen present in the unbound form of hydroxymethylmexiletine;

X is (—NH—), (—O—), or absent;

Each occurrence of R_{AA} is independently a natural or non-natural amino acid side chain;

n₁ is an integer selected from 0 to 16;

Each occurrence of n₂ is independently an integer selected from 1 to 9;

Each occurrence of R₃ is independently selected from hydrogen, a substituted alkyl group or an unsubstituted alkyl group;

Each occurrence of R₄ and R₅ is independently selected from hydrogen,

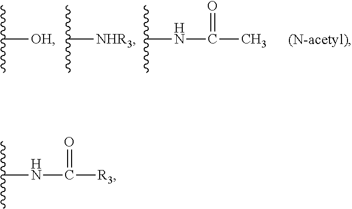

a substituted alkyl group and an unsubstituted alkyl group; In the case of a double bond, R₄ is present and R₅ is absent on the carbons that form the double bond;

In the case of a double bond in the carbon chain defined by n₁, R₄ is present and R₅ is absent on the carbons that form the double bond; and

at least R₁ is

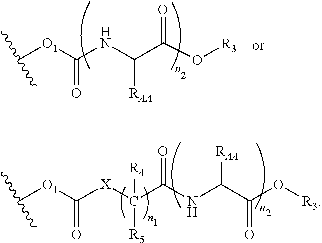

or alternatively, at least R₂ is

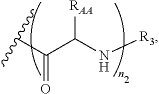

In one dicarboxylic acid linker embodiment, n₁ is 0, 1, 2, 3 or 4.

In one embodiment, each occurrence of n₂ is selected from 1, 2, 3, 4 and 5.

In a preferred embodiment, the compound of the present invention has one prodrug moiety, and the prodrug moiety has one, two or three amino acids (i.e., n₂ is 1, 2 or 3), while R₃ is H.

In another embodiment, n₂ is 1. In yet another embodiment, n₂ is 2.

In yet another embodiment, each occurrence of n₂ is 1 or 2 and each occurrence of R_{AA} is independently a natural amino acid side chain.

In one embodiment, the prodrug moiety is a homopolymer of arginine or lysine, or a heteropolymer of arginine and lysine. In a further embodiment, there are 2, 3, 4, 5, 6 or 7 amino acids in the homopolymer or the heteropolymer (i.e., n₂ is 2, 3, 4, 5, 6 or 7).

The para and meta substituted prodrug moieties described herein (e.g., prodrugs of Formulae II-XIII) can also be at a methyl group on the aromatic ring. In these embodiments, the prodrug moiety at the para or meta position is substituted with a hydrogen, while the prodrug moiety is attached to a methyl group on the aromatic ring. An example is shown below, for a carbamate linked hydroxymethylmexiletine prodrug (Formula XV).

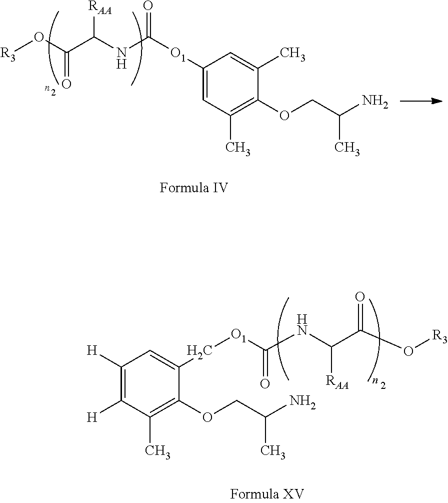

For each of the Formula (II) to (XV), the various embodiments described in relation to Formula (I) also apply (where chemically possible and relevant) but have simply been omitted for brevity from the discussion of each of Formulae (II) to (XV).

Representative Amino Acids and Peptides for Use with the Present Invention

The representative prodrugs described below are directed to p-OH mexiletine prodrugs. However, the same amino acid and peptide prodrug moieties can be used for m-OH and hydroxymethyl mexiletine prodrugs.

In the case of the phenolic function, the p-OH mexiletine metabolite may be linked to an amino acid or peptide by a carbamate or a dicarboxylic acid linker (substituted or unsubstituted). For example, malonic acid, succinic acid or glutaric acid may be used as a linker in the present invention. Other dicarboxylic acids amenable for use with the present invention are given in Tables 2 and 3. Amongst the preferred amino acids for use with the p-OH metabolite are valine, leucine or isoleucine or similar, attached either as a single amino acid or as a portion of a dipeptide. For example, dipeptides valine-valine, valine-leucine, valine-isoleucine, leucine-leucine, leucine-valine, leucine-isoleucine, isoleucine-isoleucine, isoleucine-valine, valine-isoleucine and isoleucine-leucine, can be employed. Additionally non-natural amino acids such as para-amino benzoic acid may be used alone or in conjunction with natural amino acids.

Without wishing to be bound to any particular theory, it is believed that the amino acid or peptide portion of the mexiletine or p-OH, m-OH or hydroxymethyl mexiletine prodrug selectively exploits the inherent di- and tripeptide transporter Pept1 within the digestive tract. Once absorbed, the prodrugs are subject to hydrolysis, releasing the active drug into the systemic circulation. Avoidance of direct contact between active drug and gut wall minimizes the risk of emesis while the assisted absorption of the prodrug by Pept1 ensures more consistent plasma drug levels.

A preferred embodiment of the mexiletine prodrug of Formula I is the trifluoroacetate salt of mexiletine lysine amide (Common Name: 2,6-diamino-hexanoic acid [2-(2,6-dimethylphenoxy)-1-methylethyl]-amide ditrifluoroacetate), or p-OH mexiletine lysine amide.

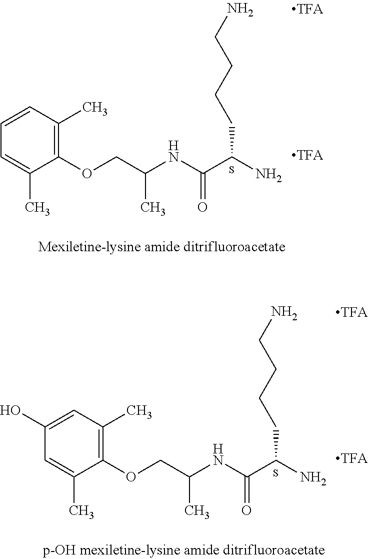

In another embodiment, the mexiletine prodrug of Formula I is the HCl salt of mexiletine glutamic acid amide (Common Name: (S)-4-Amino-4-[2-(2,6-dimethyl-phenoxy)-1-methyl-ethylcarbamoyl]-butyric acid hydrochloride) or the corresponding prodrug of the active metabolite p-OH mexiletine

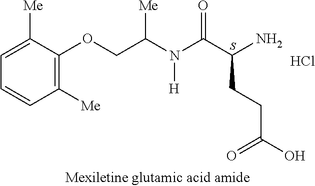

In another embodiment, the mexiletine prodrug of Formula I is the HCl salt of mexiletine glutamine amide (Common Name: (S)-2-Amino-pentanedioic acid 5-amide 1-{[2-(2,6-dimethyl-phenoxy)-1-methyl-ethyl]-amide}hydrochloride) or the corresponding prodrug of the active metabolite p-OH mexiletine

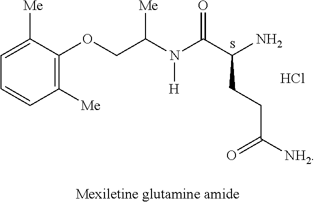

In another embodiment, the mexiletine prodrug of Formula I is the HCl salt of mexiletine homoarginine amide (Common Name: (S)-2-Amino-6-guanidino-hexanoic acid [2-(2,6-dimethyl-phenoxy)-1-methyl-ethyl]-amide dihydrochloride, or the corresponding prodrug of the active metabolite p-OH mexiletine

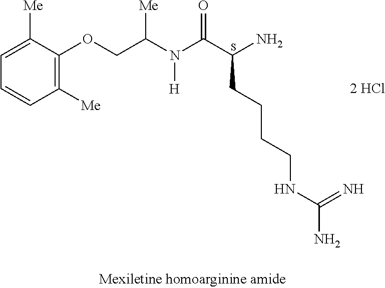

In another embodiment, the mexiletine prodrug of Formula I is the HCl salt of mexiletine methyl methionine chloride amide (Common Name: (S)-2-Amino-N-[2-(2,6-dimethyl-phenoxy)-1-methyl-ethyl]-4-(dimethyl-λ⁴-sulfanyl chloride)-butyramide hydrochloride), or the corresponding prodrug of the active metabolite p-OH mexiletine

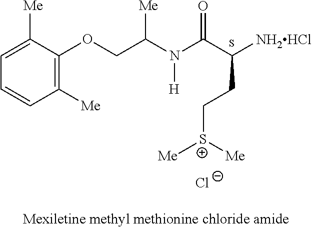

Other embodiments of single amino acid conjugates include amide conjugates with citrulline or ornithine. Amongst the preferred embodiments of the dipeptides are conjugates comprising hetero or homodimers of the aforementioned amino acids.

Oligopeptides of mexiletine can be created by attachment of polymers of any of the aforementioned amino acids to mexiletine lysine amide; mexiletine arginine amide, mexiletine citrulline amide or mexiletine ornithine amide. Alternatively, other natural or non-natural amino acids can be directly linked to mexiletine or its active metabolite. Some examples of mexiletine prodrugs are shown below. The first amino acid recited is the one bound to mexiletine via an amide linkage.

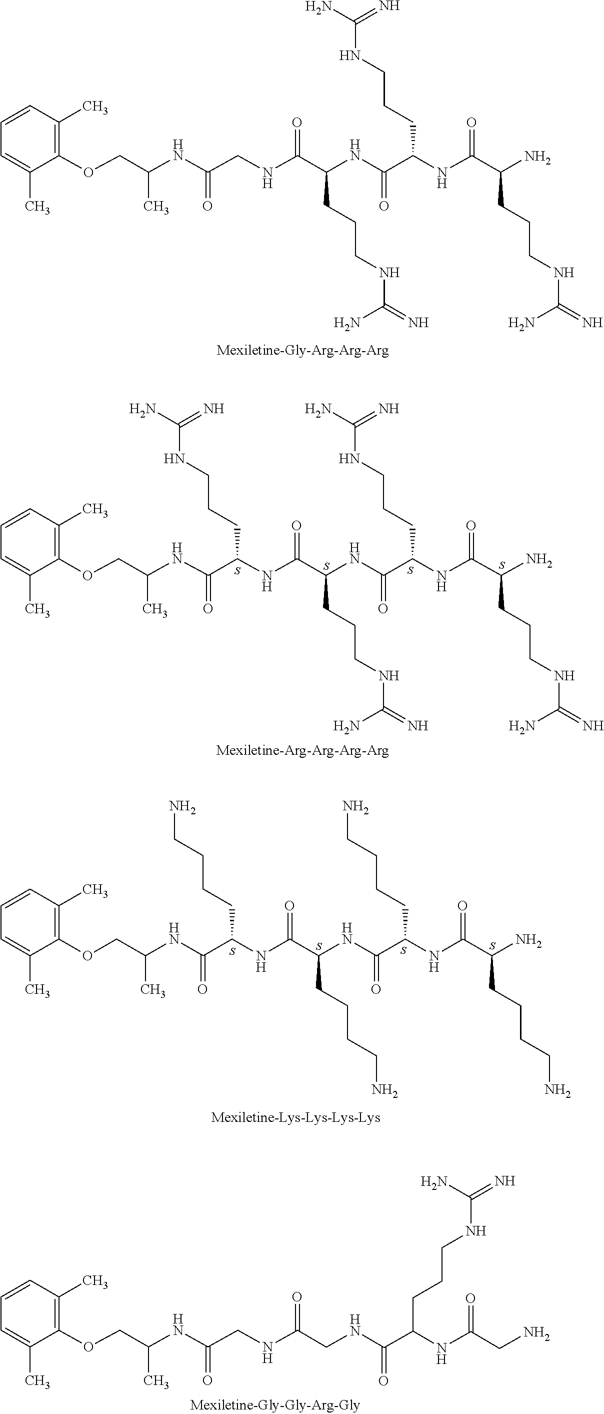

Preferred embodiments of the p-OH metabolite prodrugs include compounds with one or both of the following two single amino acids (valine and glycine), shown below:

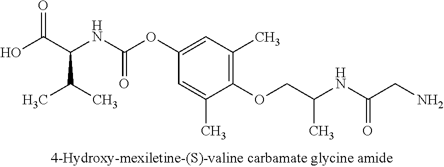

Other single amino acids in place of valine include, but are not limited to, isoleucine or tyrosine, while amino acids replacing glycine can be, but are not limited to, ornithine, citrulline or arginine amides. Non-natural amino acids carbamate conjugates include para amino benzoic acid.

Oligopeptides of the amide linked amino acids may be similar for the p-OH metabolite to those for mexiletine itself, as described above.

The preferred amino acids are all in the L configuration, however, the present invention also contemplates prodrugs of Formula I (or Formulae II-Xv) comprised of amino acids in the D configuration, and mixtures of amino acids in the D and L configurations.

Salts, Solvates, Stereoisomers, Derivatives of the Compounds of the Invention

The representative salts described below are directed to mexiletine and prodrugs of mexiletine metabolites (e.g., p-OH, m-OH mexiletine or hydroxymethylmexiletine).

The methods of the present invention further encompass the use of salts, solvates, stereoisomers of the prodrugs of mexiletine/mexiletine metabolites described herein, for example salts of the prodrugs of Formula I and Formulae II-XV given above. In one embodiment, the invention disclosed herein is meant to encompass all pharmaceutically acceptable salts of mexiletine prodrugs.

Typically, a pharmaceutically acceptable salt of a prodrug of mexiletine used in the practice of the present invention is prepared by reaction of the prodrug with a desired acid as appropriate. In the case of the p-OH mexiletine metabolite prodrug this could alternatively involve making a salt of the free carboxylic function. The salt may precipitate from solution and be collected by filtration or may be recovered by evaporation of the solvent. For example, an aqueous solution of an acid such as hydrochloric acid may be added to an aqueous suspension of the prodrug and the resulting mixture evaporated to dryness (lyophilized) to obtain the acid addition salt as a solid. Alternatively, the prodrug may be dissolved in a suitable solvent, for example, an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The acid addition salts of the prodrugs may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts of prodrugs of the p-OH mexiletine metabolite are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine and N-methylglucamine.

The base addition salts of the acidic compounds are prepared by contacting the free acid form with a sufficient amount of the desired base to produce the salt in the conventional manner. The free acid form may be regenerated by contacting the salt form with an acid and isolating the free acid.

Compounds useful in the practice of the present invention of the p-OH mexiletine metabolite may have both a basic and an acidic center and may therefore be in the form of zwitterions.

Those skilled in the art of organic chemistry will appreciate that many organic compounds can form complexes, i.e., solvates, with solvents in which they are reacted or from which they are precipitated or crystallized, e.g., hydrates with water. The salts of compounds useful in the present invention may form solvates such as hydrates useful therein. Techniques for the preparation of solvates are well known in the art (see, e.g., Brittain, Polymorphism in Pharmaceutical solids. Marcel Decker, New York, 1999). The compounds useful in the practice of the present invention can have one or more chiral centers and, depending on the nature of individual substituents, they can also have geometrical isomers.

Individual isomers of the mexiletine (or mexiletine metabolite) prodrugs described herein may be used to practice the present invention. The description or naming of a particular compound in the specification and claims is intended to include both individual enantiomers of the prodrug, as well as mixtures of enantiomers (racemic or otherwise) of the prodrug. Methods for the determination of stereochemistry and the resolution of stereoisomers are well-known in the art.

The invention thus encompasses any tautomeric forms of the compounds of Formula (I) as well as geometrical and optical isomers. Thus, it is contemplated that the present invention specifically includes tautomers of Formula (I) or pharmaceutical salts thereof.

Pharmaceutical Compositions of the Invention

While it is possible that, for use in the methods of the invention, the prodrug may be administered as the bulk substance, it is preferable to present the active ingredient in a pharmaceutical formulation, e.g., wherein the agent is in admixture with a pharmaceutically acceptable carrier selected with regard to the intended route of administration and standard pharmaceutical practice. In some embodiments of the invention, a composition of the present invention comprises a prodrug selected from a prodrug of Formulae I-XV, or a pharmaceutically acceptable salt thereof, and a pharmaceutically acceptable excipient.

The formulations of the invention may be immediate-release dosage forms, i.e. dosage forms that release the prodrug at the site of absorption immediately, or controlled-release dosage forms, i.e., dosage forms that release the prodrug over a predetermined period of time. Controlled release dosage forms may be of any conventional type, e.g., in the form of reservoir or matrix-type diffusion-controlled dosage forms; matrix, encapsulated or enteric-coated dissolution-controlled dosage forms; or osmotic dosage forms. Dosage forms of such types are disclosed, for example, in Remington, The Science and Practice of Pharmacy, 20^{th} Edition, 2000, pp. 858-914. The formulations of the present invention can be administered from one to six times daily, depending on the dosage form and dosage.

Absorption of amino acid and peptide prodrugs of mexiletine/p-OH mexiletine metabolite is likely to proceed via an active transporter such as Pept1. This transporter is believed to be largely confined to the upper GI tract and as such limits the utility of conventional sustained release formulations for continued absorption along the whole length of the GI tract. For those prodrugs of mexiletine/mexiletine metabolite which do not result in sustained plasma drugs levels due to continuous systemic generation of active form, a plasma reservoir of prodrug, gastroretentive or mucoretentive formulations analogous to those used in metformin products such as Glumetz® or Gluphage XR® may be useful. The former exploits a drug delivery system known as Gelshield Diffusion™ Technology while the latter uses a so-called Acuform™ delivery system. In both cases the concept is to slow drug delivery into the ileum maximizing the period over which absorption take place and effectively prolonging plasma drug levels. Other drug delivery systems affording delayed progression along the GI tract may also be of value.

For those mexiletine prodrugs that do not require the sophistication of the aforementioned delivery systems conventional formulations as described below should be adequate.

In one aspect, the present invention provides a pharmaceutical composition comprising at least one active pharmaceutical ingredient (i.e., a prodrug of mexiletine or a mexiletine metabolite), or a pharmaceutically acceptable derivative (e.g., a salt or solvate) thereof, and a pharmaceutically acceptable carrier. In particular, the invention provides a pharmaceutical composition comprising a therapeutically effective amount of at least one prodrug of the present invention, or a pharmaceutically acceptable derivative thereof, and a pharmaceutically acceptable carrier.

For the methods of the invention, the prodrug employed in the present invention may be used in combination with other therapies and/or active agents. Accordingly, the present invention provides, in a further aspect, a pharmaceutical composition comprising at least one compound useful in the practice of the present invention, or a pharmaceutically acceptable salt or solvate thereof, a second active agent, and, optionally a pharmaceutically acceptable carrier.

When combined in the same formulation it will be appreciated that the two compounds are preferably stable in the presence of and compatible with each other and the other components of the formulation. When formulated separately they may be provided in any convenient formulation, conveniently in such manner as are known for such compounds in the art. In some embodiments, the two compounds are either (1) two distinct prodrugs of mexiletine, (2) a prodrug of mexiletine and a prodrug of p-OH mexiletine, (3) two prodrugs of p-OH mexiletine, (4) a prodrug of mexiletine and a prodrug of m-OH mexiletine, (5) a prodrug of mexiletine and a prodrug of hydroxymethylmexiletine, (6) two prodrugs of m-OH mexiletine, (7) two prodrugs of hydroxymethylmexiletine, (8) a prodrug of meta-OH mexiletine and a prodrug of p-OH mexiletine or (9) a prodrug of hydroxymethylmexiletine and a prodrug of p-OH mexiletine. In other embodiments, the two compounds include a prodrug of Formula I and another compound for a distinct indication.

The prodrugs used herein may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro edit. 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may also be used.

The compounds used in the invention may be milled using known milling procedures such as wet milling to obtain a particle size appropriate for tablet formation and for other formulation types. Finely divided (nanoparticulate) preparations of the compounds may be prepared by processes known in the art, for example see International Patent Application No. WO 02/00196 (SmithKline Beecham).

The compounds and pharmaceutical compositions of the present invention are intended to be administered orally (e.g., as a tablet, sachet, capsule, pastille, pill, bolus, powder, paste, granules, bullets or premix preparation, ovule, elixir, solution, suspension, dispersion, gel, syrup or as an ingestible solution). In addition, compounds may be present as a dry powder for constitution with water or other suitable vehicle before use, optionally with flavoring and coloring agents. Solid and liquid compositions may be prepared according to methods well-known in the art. Such compositions may also contain one or more pharmaceutically acceptable carriers and excipients which may be in solid or liquid form.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium aluminum silicate, polyethylene glycol or bentonite.

Examples of pharmaceutically acceptable fillers for oral compositions useful herein include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes for the oral compositions include, but are not limited to, synthetic and natural dyes such as titanium dioxide, beta-carotene and extracts of grapefruit peel.

Examples of useful pharmaceutically acceptable coatings for the oral compositions, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions include, but are not limited to, aspartame, saccharin, saccharin sodium, sodium cyclamate, xylitol, mannitol, sorbitol, lactose and sucrose.

Suitable examples of pharmaceutically acceptable buffers useful herein include, but are not limited to, citric acid, sodium citrate, sodium bicarbonate, dibasic sodium phosphate, magnesium oxide, calcium carbonate and magnesium hydroxide.

Suitable examples of pharmaceutically acceptable surfactants useful herein include, but are not limited to, sodium lauryl sulfate and polysorbates.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Suitable examples of pharmaceutically acceptable stabilizers and antioxidants include, but are not limited to, ethylenediaminetetriacetic acid (EDTA), thiourea, tocopherol and butyl hydroxyanisole.

The pharmaceutical compositions of the invention may contain from 0.01 to 99% weight per volume of the prodrugs encompassed by the present invention

Dosages

The prodrug dosages provided herein refer to the amount of mexiletine free base equivalents, unless otherwise indicated.

Appropriate subjects to be treated according to the methods of the invention include any human or animal in need of such treatment. Methods for the diagnosis and clinical evaluation of pain, including the severity of the pain experienced by an animal or human are well known in the art. Thus, it is within the skill of the ordinary practitioner in the art (e.g., a medical doctor or veterinarian) to determine if a patient is in need of treatment for pain. The patient is preferably a mammal, more preferably a human, but can be any animal, including a laboratory animal in the context of a clinical trial or screening or activity experiment employing an animal model. Thus, as can be readily appreciated by one of ordinary skill in the art, the methods and compositions of the present invention are particularly suited to administration to any animal, particularly a mammal, and including, but by no means limited to, domestic animals, such as feline or canine subjects, farm animals, such as but not limited to bovine, equine, caprine, ovine, and porcine subjects, research animals, such as mice, rats, rabbits, goats, sheep, pigs, dogs, cats, etc., avian species, such as chickens, turkeys, songbirds, etc.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Mexitil®, the FDA approved mexiletine hydrochloride formulation, is available in 150 mg, 200 mg and 250 mg capsules. 100 mg of mexiletine hydrochloride is equivalent to 83.31 mg of mexiletine base. Typically, Mexitil® is administered every 8 hours. In one embodiment of the invention, the prodrug dose is selected from one of the doses of Mexitil', and can be administered once every eight hours. In another embodiment, the prodrug dose is selected from one of the doses of Mexitil®, and can be administered once every twelve or twenty four hours

In one embodiment, an effective daily dose of the mexiletine prodrug is from 1 mg to 2000 mg, preferably from 100 mg to 2000 mg, of the prodrug. For example, the prodrugs encompassed by the present invention may be formulated in a dosage form that provides from about 200 mg to about 2000 mg of the prodrug per day, preferably from about 200 mg to about 1000 mg of the prodrug per day. In a preferred embodiment, an effective amount of the a prodrug of the present invention is either 250 mg, 500 mg, 750 mg, /day.

In another embodiment, an effective daily dose of the p-OH mexiletine prodrug is from 4 mg to 8000 mg, preferably from 400 mg to 8000 mg, of the prodrug. In an alternative embodiment, an effective daily amount of the p-OH mexiletine prodrug is either 1000 mg or 3000 mg.

Depending on the severity of pain to be treated, a suitable therapeutically effective and safe dosage, as may readily be determined within the skill of the art, and without undue experimentation, may be administered to subjects. For oral administration to humans, the daily dosage level of the prodrug may be in single or divided doses. The duration of treatment may be determined by one of ordinary skill in the art, and should reflect the nature of the pain (e.g., a chronic versus an acute condition) and/or the rate and degree of therapeutic response to the treatment.

In the methods of treating pain, the prodrugs encompassed by the present invention may be administered in conjunction with other therapies and/or in combination with other active agents. For example, the prodrugs encompassed by the present invention may be administered to a patient in combination with other active agents used in the management of pain. An active agent to be administered in combination with the prodrugs encompassed by the present invention may include, for example, a drug selected from the group consisting of non-steroidal anti-inflammatory drugs including acetaminophen and ibuprofen or opioids including oxycodone, oxymorphone, levorphanol, or anti-emetic agents such as ondanstron, domerperidone, hyoscine or metoclopramide. In such combination therapies the prodrugs encompassed by the present invention may be administered prior to, concurrent with, or subsequent to the other therapy and/or active agent.

Where the prodrugs encompassed by the present invention are administered in conjunction with another active agent, the individual components of such combinations may be administered either sequentially or simultaneously in separate or combined pharmaceutical formulations by any convenient route. When administration is sequential, either the prodrugs encompassed by the present invention or the second active agent may be administered first. For example, in the case of a combination therapy with another active agent, the prodrugs encompassed by the present invention may be administered in a sequential manner in a regimen that will provide beneficial effects of the drug combination. When administration is simultaneous, the combination may be administered either in the same or different pharmaceutical compositions. For example, the prodrugs encompassed by the present invention and another active agent may be administered in a substantially simultaneous manner, such as in a single capsule or tablet having a fixed ratio of these agents or in multiple, separate capsules or tablets for each agent.

When the prodrugs encompassed by the present invention are used in combination with another agent active in the methods for treating pain, the dose of each compound may differ from that when the compound is used alone. Appropriate doses will be readily appreciated by those skilled in the art.

Methods of the Invention

One embodiment of the present invention is a method of treating a disorder in a subject in need thereof with mexiletine. The method comprises orally administering a mexiletine prodrug of the present invention, pharmaceutically acceptable salt thereof, or composition thereof, to a subject in need thereof, wherein the mexiletine prodrug is comprised of mexiletine or a mexiletine metabolite (e.g., p-OH, m-OH mexiletine or hydroxymethylmexiletine) covalently bonded to at least one amino acid or peptide of 2-9 amino acids in length. The mexiletine prodrug has one or two prodrug moieties. One prodrug moiety can be bound to the amino group of mexiletine or a mexiletine metabolite through a peptide bond. Alternatively or in addition, the mexiletine metabolite (e.g., p-OH, m-OH mexiletine or hydroxymethylmexiletine) can have a prodrug moiety bound to it through a carbamate or dicarboxylic acid linker at the phenolic oxygen. The amount of the mexiletine is preferably a therapeutically effective amount (e.g., an analgesic effective amount). The disorder may be one treatable with mexiletine. For example, the disorder may be neuropathic pain or arrhythmia.

In a further embodiment of the invention, a method is provided for treating a disorder in a subject in need thereof with mexiletine, without inducing GI side effects associated with mexiletine. The method comprises orally administering a mexiletine prodrug of the present invention, pharmaceutically acceptable salt thereof, or composition thereof, to a subject in need thereof, wherein the mexiletine prodrug is comprised of mexiletine or a mexiletine metabolite (e.g., p-OH, m-OH mexiletine or hydroxymethylmexiletine) covalently bonded to at least one amino acid or peptide of 2-9 amino acids in length, and wherein upon oral administration, the prodrug or pharmaceutically acceptable salt minimizes, if not completely avoids, the gastrointestinal side effects usually seen after oral administration of the unbound mexiletine. The mexiletine prodrug has one or two prodrug moieties. One prodrug moiety can be bound to the amino group of mexiletine or a mexiletine metabolite through a peptide bond. Alternatively or in addition, the mexiletine metabolite (e.g., p-OH, m-OH mexiletine or hydroxymethylmexiletine) can have a prodrug moiety bound to it through a carbamate or dicarboxylic acid linker at the phenolic oxygen. The amount of the mexiletine is preferably a therapeutically effective amount (e.g., an analgesic effective amount). The disorder may be one treatable with mexiletine. For example, the disorder may be neuropathic pain or arrhythmia. In a further embodiment, the GI side effect associated with administration of mexiletine is selected from, but is not limited to, emesis, nausea and abdominal discomfort.

The mexiletine prodrugs described herein may induce statistically significant lower average (e.g., mean) adverse effects on gut motility in the gastrointestinal environment as compared to a non-prodrug mexiletine salt form such as mexiletine HCl.

In an alternative aspect of the invention, a method for improving the pharmacokinetics and extending the duration of action of mexiletine in a subject in need thereof is provided. The method comprises administering to a subject in need thereof an effective amount of a prodrug of the present invention, or a composition thereof, wherein the plasma concentration time profile is modulated to minimize an initial upsurge in concentration of mexiletine, minimizing any unwanted effects, while significantly extending the time for which the drug persists in plasma (resulting from continuing generation from the prodrug) and hence duration of action.

In a further aspect, a method for reducing inter- or intra-subject variability of mexiletine plasma levels is provided. The method comprises administering to a subject, or group of subjects in need thereof, an effective amount of a prodrug of the present invention, or a composition thereof.

In a further embodiment, a prodrug of p-OH or m-OH mexiletine is used in the method.

In another embodiment, a method is provided for eliminating, reducing or treating neuropathic pain. The method comprises orally administering a mexiletine prodrug of the present invention, pharmaceutically acceptable salt thereof, or composition thereof, to a subject in need thereof, wherein the mexiletine prodrug is comprised of mexiletine or a mexiletine metabolite (e.g., p-OH, m-OH mexiletine or hydroxymethylmexiletine) covalently bonded to at least one amino acid or peptide of 2-9 amino acids in length. The mexiletine prodrug has one or two prodrug moieties. One prodrug moiety can be bound to the amino group of mexiletine or a mexiletine metabolite through a peptide bond. Alternatively or in addition, the mexiletine metabolite (e.g., p-OH, m-OH mexiletine or hydroxymethylmexiletine) can have a prodrug moiety bound to it through a carbamate or dicarboxylic acid linker at the phenolic oxygen. The amount of the mexiletine is preferably a therapeutically effective amount (e.g., an analgesic effective amount).

In a further embodiment, a prodrug of p-OH or m-OH mexiletine is used in the method.

Another embodiment of the invention is directed to reducing the inter- and intra-subject variability of mexiletine serum levels. The method comprises orally administering a mexiletine prodrug of the present invention, pharmaceutically acceptable salt thereof, or composition thereof, to a subject in need thereof, wherein the mexiletine prodrug is comprised of mexiletine or a mexiletine metabolite (e.g., p-OH, m-OH mexiletine or hydroxymethylmexiletine) covalently bonded to at least one amino acid or peptide of 2-9 amino acids in length. The mexiletine prodrug has one or two prodrug moieties. One prodrug moiety can be bound to the amino group of mexiletine or a mexiletine metabolite through a peptide bond. Alternatively or in addition, the mexiletine metabolite (e.g., p-OH, m-OH mexiletine or hydroxymethylmexiletine) can have a prodrug moiety bound to it through a carbamate or dicarboxylic acid linker at the phenolic oxygen. The amount of the mexiletine is preferably a therapeutically effective amount (e.g., an analgesic effective amount).

Yet another embodiment of the invention related to increasing the reproducibility of the bioavailability of mexiletine, in a subject in need thereof. The method comprises orally administering a mexiletine prodrug of the present invention, pharmaceutically acceptable salt thereof, or composition thereof, to a subject in need thereof, wherein the mexiletine prodrug is comprised of mexiletine or a mexiletine metabolite (e.g., p-OH, m-OH mexiletine or hydroxymethylmexiletine) covalently bonded to at least one amino acid or peptide of 2-9 amino acids in length. The mexiletine prodrug has one or two prodrug moieties. One prodrug moiety can be bound to the amino group of mexiletine or a mexiletine metabolite through a peptide bond. Alternatively or in addition, the mexiletine metabolite (e.g., p-OH, m-OH mexiletine or hydroxymethylmexiletine) can have a prodrug moiety bound to it through a carbamate or dicarboxylic acid linker at the phenolic oxygen. The amount of the mexiletine is preferably a therapeutically effective amount (e.g., an analgesic effective amount).

In a further embodiment, a prodrug of p-OH mexiletine is used in the method.

The present invention also includes the synthesis of all pharmaceutically acceptable isotopically-labelled compounds of Formula (I) wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labelled compounds can generally be prepared by conventional techniques known to those skilled in the art or by processes analogous to those described using an appropriate isotopically-labelled reagent in place of the non-labeled reagent previously employed.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### EXAMPLES

The present invention is further illustrated by reference to the following Examples. However, it should be noted that these Examples, like the embodiments described above, are illustrative and are not to be construed as restricting the enabled scope of the invention in any way.

General Synthesis Procedures

The synthesis of a mexiletine prodrug of the present invention can be achieved in two distinct steps. An activated ester of an amino acid or peptide, for example, the activated ester of (S)-lysine, N,N′-di-t-butyloxycarbonyl-(S)-lysine succinimide, can be coupled to (rac)-mexiletine hydrochloride to yield the N-protected prodrug, (rac)-mexiletine-N,N′-di-t-butyloxycarbonyl-(S)-lysine. The compound can then be deprotected with trifluoroacetic acid to yield the prodrug.

As stated above, the activated lysine can be readily substituted for another activated amino acid or peptide

### Example 1

### Synthesis of (rac)-mexiletine-(S)-lysine ditrifluoroacetate

The synthesis of mexiletine-(S)-lysine-ditrifluoroacetate was achieved in two distinct steps. Initially, the activated ester of (S)-lysine, N,N′-di-t-butyloxycarbonyl-(S)-lysine succinimide, was coupled to (rac)-mexiletine hydrochloride in the presence of N-methylmorpholine (NMM) to yield the N-protected prodrug, (rac)-mexiletine-N,N′-di-t-butyloxycarbonyl-(S)-lysine, after purification by chromatography (Scheme I).

Subsequent deprotection of the BOC groups was then achieved using trifluoroacetic acid to give the desired (rac)-mexiletine-(S)-lysine-ditrifluoroacetate as a viscous glassy oil. The oil was found to foam on drying under high vacuum, but collapsed on standing in air. For the purposes of clarity, only one enantiomer of mexiletine is shown.

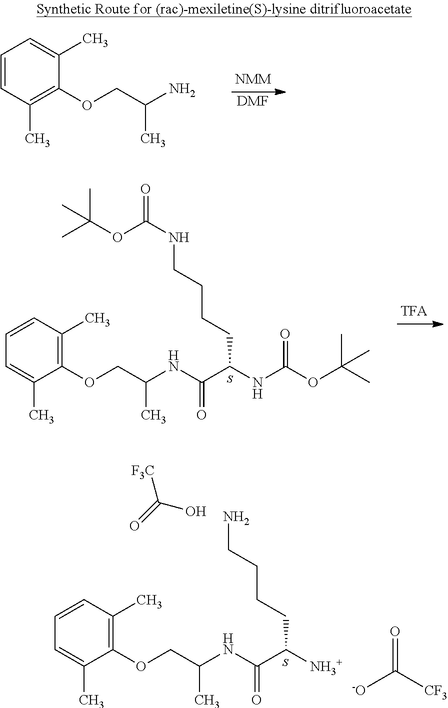

¹H NMR (DMSO-d₆) spectrum

8.60 (m, 1H, NH), 8.16 (br, 3H, NH₃⁺), 7.76 (br, 3H, NH₃⁺), 7.02 (m, 2H, ArH), 6.92 (m, 1H, ArH), 4.22 (m, 1H, □-CH), 3.67 (d, J=4.5 Hz, CH₂), 2.73 (m, 2H, NCH₂), 2.21 (s, 6H, 2×CH₃), 1.73 (m, 2H, CH₂), 1.52 (m, 2H, CH₂), 1.30 (m, 5H, CH₃+CH₂).

### Example 2

### Synthesis of (rac)-mexiletine-glycine trifluoroacetate

N-t-butyloxycarbonyl-glycine succinimide was coupled to (rac)-mexiletine hydrochloride in the presence of NMM, to yield the N-protected prodrug, (rac)-mexiletine-N-t-butyloxycarbonyl-glycine in good yield after purification by chromatography (Scheme 2).

Subsequent deprotection of the BOC groups was then achieved using trifluoroacetic acid. Trituration with diethyl ether and filtration gave the required (rac)-mexiletine glycine trifluoroacetate as a white solid in excellent yield (scheme 2). Note, for the purposes of clarity, only one enantiomer of mexiletine is shown in scheme 2.

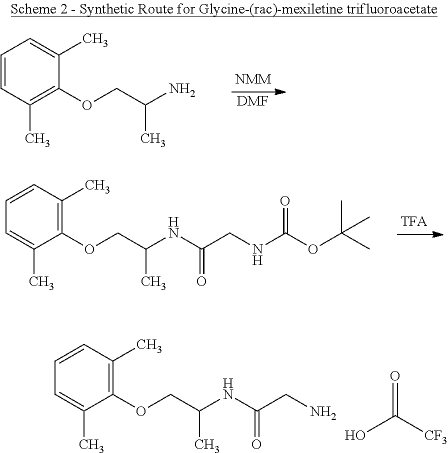

Subsequent deprotection of the BOC groups was achieved using trifluoroacetic acid and filtration from diethyl ether give glycine-(rac)-mexiletine trifluoroacetate as a white solid in excellent yield.

¹H NMR (DMSO-d₆) spectrum

8.52 (d, J=7.8 Hz, 1H, NH), 8.03 (br, 3H, NH₃⁺), 7.01 (m, 2H, ArH), 6.93 (m, 1H, ArH), 3.64 (m, 4H, 2×CH₂), 2.22 (s, 6H, 2×CH₃), 1.28 (d, J=6.6 Hz, 3H, CH₃).

### Example 3

### Synthesis of mexiletine-(S)-homoarginine amide dihydrochloride

The synthesis of mexiletine-(S)-homoarginine amide dihydrochloride was accomplished in four distinct steps. The ‘activated ester’ N-Boc-(S)-homoarginine-(NO₂) N-hydroxysuccinimide ester was made via a DCC coupling between N-hydroxysuccinimide and N-Boc-(S)-homoarginine-(NO₂). Subsequent reaction with mexiletine hydrochloride yielded the N-protected prodrug, N-Boc-(5)-homoarginine-(NO₂)-mexiletine in good yield after purification using a Biotage Isolera automated chromatography system under reversed-phase conditions.

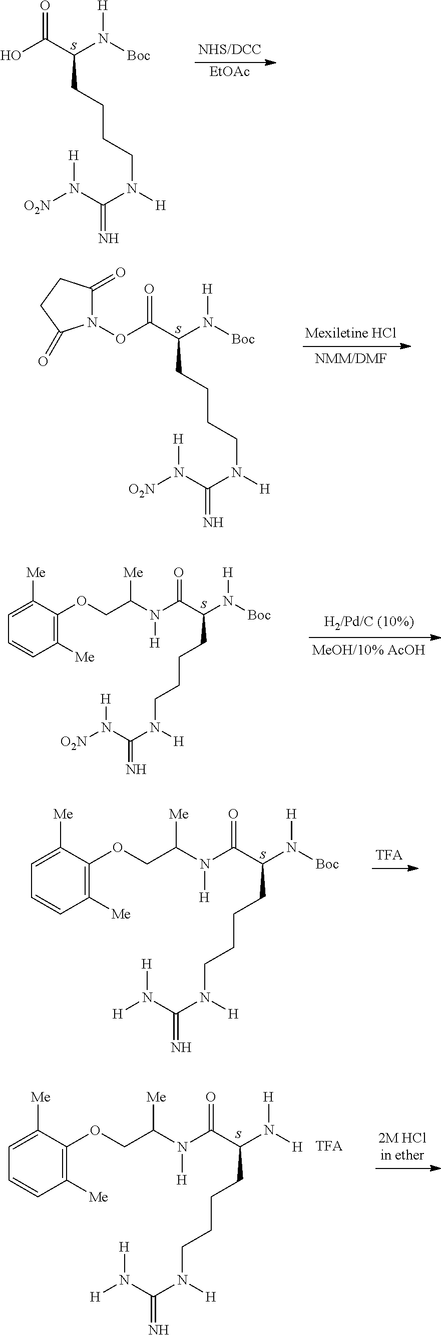

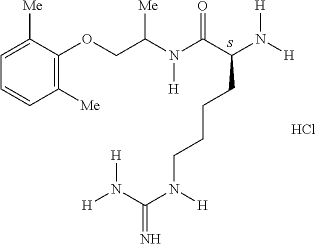

### Synthetic route for mexiletine-(S)-homoarginine amide dihydrochloride

The nitro-group was reduced via catalytic hydrogenation using palladium on carbon to give N-Boc-(5)-homoarginine-mexiletine. Removal of the Boc group was accomplished with trifluoroacetic acid. The crude product was subjected to salt exchange with 2M hydrogen chloride in diethyl ether and purified using a Biotage Isolera chromatography system under reversed-phase conditions to afford mexiletine-(5)-homoarginine amide dihydrochloride, as a white glassy solid

¹H NMR (DMSO-d₆) spectrum

8.79 (d, J=8.1 Hz, 1H, NH), 8.35 (br, 3H, NH₃⁺), 7.92 (m, 1H, NH), 7.02 (d, J=7.5 Hz, 2H, 2×ArH), 6.91 (m, 1H, ArH), 4.20 (m, 1H, □-CH), 3.82-3.65 (m, 3H, CH+OCH₂), 3.09 (m, 2H, □-CH₂), 2.22 (s, 6H, 2×CH₃), 1.75 (m, 2H, CH₂), 1.49-1.37 (m, 4H, 2×CH₂), 1.28 (m, 3H, CH₃).

### Example 4

### Synthesis of mexiletine-(S)-glutamic acid amide hydrochloride

The synthesis of mexiletine-(5)-glutamic acid amide hydrochloride was achieved in two distinct steps. Initially, the ‘activated ester’ of (S)-glutamic acid, N-Boc-(S)-glutamic acid (tert-butyl ester) N-hydroxysuccinimide ester, was coupled to mexiletine hydrochloride. This gave the protected prodrug, N-Boc-(S)-glutamic acid (tert-butyl ester)-mexiletine in good yield after purification by chromatography.

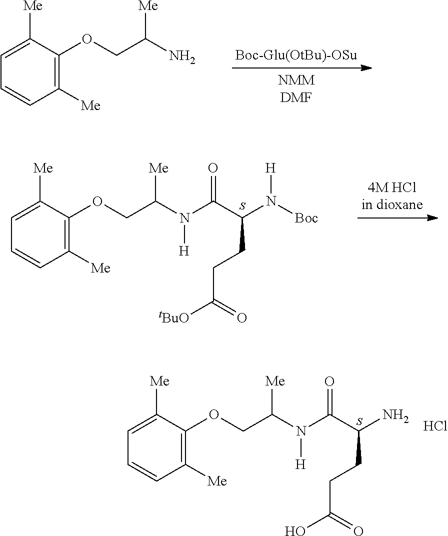

### Synthetic route for mexiletine-(S)-glutamic acid amide hydrochloride

Subsequent deprotection of the Boc and tert-butyl groups was achieved using a solution of 4M hydrogen chloride in dioxane. The crude product was purified using a Biotage Isolera automated chromatography system under reversed-phase conditions to afford the desired mexiletine-(S)-glutamic acid amide hydrochloride as a glassy white solid.

¹H NMR (DMSO-d₆) spectrum

8.71 (m, 1H, NH), 8.31 (s, 3H, NH₃⁺), 7.01 (d, J=7.5 Hz, 2H, 2×ArH), 6.91 (m, 1H, ArH), 4.21 (m, 1H, glutamic acid α-CH), 3.67 (m, 3H, obscured, mexiletine CH+OCH₂), 2.36 (m, 2H, □-CH₂), 2.23 (s, 3H, CH₃), 2.21 (s, 3H, CH₃), 1.99 (m, 2H, CH₂), 1.27 (d, J=6.6 Hz, 3H, CH₃).

### Example 5

### Mexiletine-[(S)-S-methyl-methionine chloride]amide hydrochloride

The synthesis of mexiletine-[(S)-S-methyl-methionine chloride]amide hydrochloride was achieved in three distinct steps. The ‘activated ester’ of (S)-methionine, N-Boc-(S)-methionine N-hydroxysuccinimide ester, was first coupled to mexiletine hydrochloride to yield the protected prodrug, N-Boc-(S)-methionine-mexiletine in good yield. Subsequent S-methylation was achieved using methyl iodide in methanol and the compound was purified by reversed-phase chromatography to give [N-Boc-(S)-S-methyl-methionine iodide]-mexiletine.

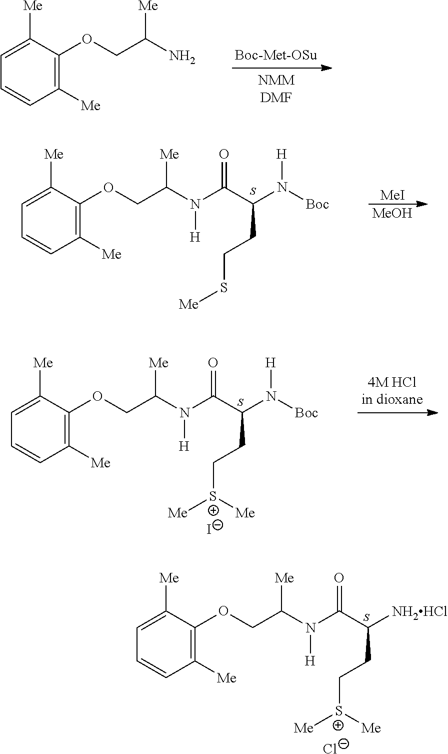

### Synthetic route for mexiletine-[(S)-S-methyl-methionine chloride]amide hydrochloride

Deprotection of the Boc group was carried out using 4 M hydrogen chloride in dioxane, followed by purification by reversed-phase chromatography, to afford the desired mexiletine-[(S)-S-methyl-methionine]amide hydrochloride.

¹H NMR (DMSO-d₆) spectrum

9.32 (bd, 1H, NH), 8.73 (br, 3H, NH₃⁺), 7.02 (d, J=7.2 Hz, 2H, 2×ArH), 6.92 (m, 1H, ArH), 4.20 (m, 1H, □-CH), 4.06 (m, 1H, CH), 3.72 (m, 4H, CH₂+OCH₂), 3.01 (s, 3H, S—CH₃), 2.98 (s, 3H, S—CH₃), 2.32 (obscured, 2H, CH₂), 2.22 (s, 6H, 2×CH₃), 1.29 (m, 3H, CH₃).

### Example 6

### Mexiletine-(S)-glutamine amide hydrochloride

The synthesis of mexiletine-(S)-glutamine amide hydrochloride was achieved in a similar manor to that for mexiletine-(S)-glutamic acid amide hydrochloride. In this case, N-Boc-(S)-glutamine N-hydroxysuccinimide ester was coupled to mexiletine hydrochloride to yield the N-protected prodrug, N-Boc-(S)-glutamine-mexiletine in good yield.

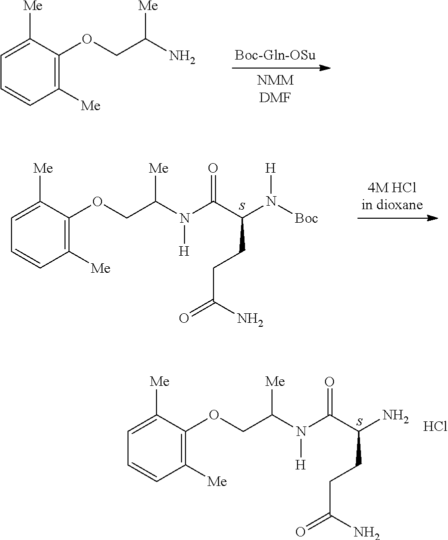

### Synthetic route for mexiletine-(S)-glutamine amide hydrochloride

Subsequent deprotection of the Boc group was achieved using 4 M hydrogen chloride in dioxane to afford mexiletine-(S)-glutamine amide hydrochloride as an off-white solid.

¹H NMR (DMSO-d₆) spectrum

8.73 (d, J=8.1 Hz, 1H, NH), 8.35 (br, 3H, NH₃⁺), 7.50 (br, 1H, 0.5 NH₂), 7.01 (d, J=7.5 Hz, 2H, 2×ArH), 6.92 (m, 2H, ArH+0.5 NH₂), 4.20 (m, 1H, α-CH), 3.66 (m, 3H, obscured, CH₂+CH), 2.21 (m, 6H, 2×CH₃), 1.97 (m, 2H, □-CH₂), 1.28 (d, J=6.6 Hz, 3H, CH₃).

Evaluation of the Compounds

It is believed that emetic activity or nausea arises as a direct result of a local anesthetic effect in the stomach. This results from inhibition of the of low wave movement (the “housekeeper” wave) in the stomach which facilitates stomach emptying. The local anesthetic effect is mediated through blockade of sodium channels. It is considered that the compounds of the present invention reduce or eliminate emesis by having very poor activity, represented by a high IC₅₀ value against sodium channels. Thus the sodium channel blocking effect of mexiletine is temporarily inactivated by administering compounds of the invention instead of mexiletine itself. Once the compounds have been absorbed, they are quantitatively converted to mexiletine, thereby providing all the therapeutic benefit recognized for mexiletine with reduced or eliminated emesis and/or nausea. The IC₅₀ values shown in the following Examples demonstrate the reduced potential for emesis of the compounds. (shown, for example, by high IC₅₀ values in Table 5).

### Example 7

### Effects of Mexiletine and Various Mexiletine Amino Acid Prodrugs on Cloned Nav1.1 Channels Expressed in Mammalian Cells

In an attempt to identify amino acid prodrugs of mexiletine which may be (transiently) inactivated and hence less likely to have a direct emetic effect within the gut, a series of conjugates were screened in vitro for their potential local anaesthetic activity by assessing their effects on the sodium 1.1 channel expressed in mammalian cells.

Methods

hNav 1.1 Test Procedures

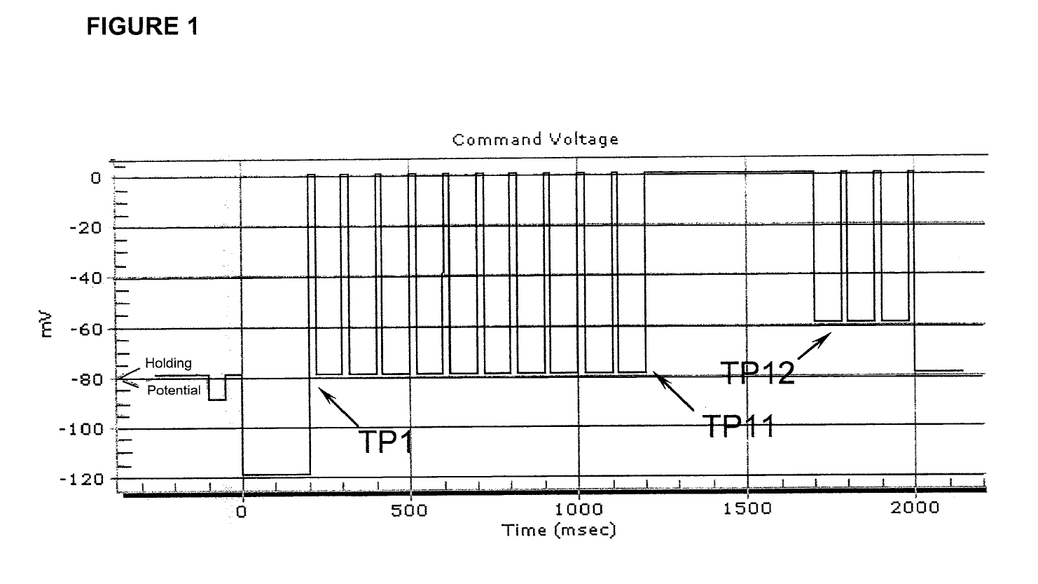

Using CHO cells stably transfected with hNav 1.1 channel cDNA (SCN1A gene), the potential block of hNav1.1 channel was measured using a stimulus voltage pattern shown in FIG. 1; voltage potentials are indicated in Table 4. The pulse pattern was repeated twice: before and 5 minutes after TA addition and peak current amplitudes at three test pulses were measured (ITP1, TP11 and ITP12).

TABLE 4
Voltage-protocol parameters for hNav1.1 channel

| | | | Test | | | |
|---|---|---|---|---|---|---|
| | | | Pulse 1-10, | Test | | |
| | Holding | Pre-Pulse | 12-14 | Pulse 11 | Interpulse | Test Pulse 1-14 |
| | Potential | Potential | Duration | Duration | Duration | Potential |
| Channel | (mV) | (mV) | (ms) | (ms) | (ms) | (mV) |
| Nav1.1 | −80 | −120 | 20 | 500 | 80 | 0 |

Data Analysis

Data acquisition and analyses was performed using the IonWorks Quattro™ system operation software (version 2.0.2; Molecular Devices Corporation, Union City, Calif.). Data was corrected for leak current.

The tonic block was calculated as:

% Block (Tonic)=(1−I_{TP1,TA}/I_{TP1,Control})×100%,

where I_{TP1, Control} and I_{TP1, TA} are the inward peak Na⁺ currents elicited by the TP1 in control and in the presence of a test article, respectively.
10 Hz Block—the frequency-dependent block at stimulation frequency 10 Hz was calculated as:

% Block (10 Hz)=(1−I_{TP11,TA}/I_{TP11,Control}))×100%,

where I_{TP11, Control} and I_{TP11, TA} are the inward peak Na⁺ currents elicited by the TP11 in control and in the presence of a test article, respectively.
The inactivation state block is defined as the decrease in test pulse (TP12) current amplitude due to the conditioning depolarizing pulse (TP11). The inactivation state block was calculated as:

% Block (inactivation state)=(1−(I_{TP12,TA}/I_{TP12,TA})×100%,

where I_{TP12, Control} and I_{TP12, TA} are the inward peak Na⁺ currents elicited by the TP12 in control and in the presence of a test article, respectively.
Concentration-response data for the blocks were fit to an equation of the following form:

% Block={1−1/[1+([Test]/IC₅₀)^{N}]}*100%,

where [Test] is the concentration of test article, IC₅₀ is the concentration of the test article producing half-maximal inhibition, N is the Hill coefficient, and % Block is the percentage of ion channel current inhibited at each concentration of the test article. Nonlinear least squares fits were solved with the Solver add-in for Excel 2000 (Microsoft, Redmond, Wash.).

Results

As can be seen in Table 5 seven of the twenty two compounds tested had IC50 values in excess of 10-fold above the parent and four had potencies ˜20-fold higher. These were the homoarginine, arginine, glutamic acid and S-methylmethionine chloride conjugates with values of 624, 811, >1000 uM and >1000 uM respectively (with the 10 Hz block) compared to 38 uM for mexiletine itself. Such reduction in potency might be expected to reduce the potential for a direct action on the stomach/gut epithelium and resultant emesis.

TABLE 5
Summary Effects of various mexiletine
prodrugs on hNav1.1 Channel

| | IC₅₀ (μM) | IC₅₀ (μM) | IC₅₀ (μM) |
|---|---|---|---|
| Compound | 10 Hz block | Tonic block | Inactivated state |
| Mexiletine methionine | 5.2 | 26 | 1.3 |
| amide | | | |
| Mexiletine pipecolic | 6.0 | 26 | 2.7 |
| acid amide | | | |
| Mexiletine | 10.3 | 30.5 | 3.2 |
| dimethylglycine amide | | | |
| Mexiletine 4- | 37 | 147 | 15.5 |
| hydroxyproline amide | | | |
| Mexiletine | 38.2 | 115 | 9.1 |
| hydrochloride | | | |
| Mexiletine sarcosine | 38.9 | 186 | 6.3 |
| amide | | | |
| Mexiletine threonine | 41.4 | 234.5 | 11.6 |
| amide | | | |
| Mexiletine histidine | 42.1 | 79.5 | 21.2 |
| amide | | | |
| Mexiletine serine amide | 50.3 | 238.5 | 14.1 |
| Mexiletine 2-methyl β | 51.8 | 137.2 | 16.3 |
| alanine amide | | | |
| Mexiletine β alanine | 59.5 | 245.3 | 23.5 |
| amide | | | |
| Mexiletine glycine | 106.9 | 397.5 | 21.2 |
| amide | | | |
| Mexiletine glutamine | 138.9 | 750.5 | 67.3 |
| amide | | | |
| Mexiletine cyclopropyl | 169.9 | 474.8 | 42.7 |
| glycine amide | | | |
| Mexiletine β amino | 179.7 | 477.7 | 61.8 |
| alanine amide | | | |
| Mexiletine nicotinic acid | 350.1 | 494.7 | 61.25 |
| amide | | | |
| Mexiletine citrulline | 358.6 | 491.1 | 180.5 |
| amide | | | |
| Mexiletine isonicotinic | 439.3 | 817.58 | 130.2 |
| acid amide | | | |
| Mexiletine homo | 623.6 | 742.5 | 257.5 |
| arginine amide | | | |
| Mexiletine arginine | 811.1 | >1000 | 328.3 |
| amide | | | |
| Mexiletine glutamic acid | >1000 | >1000 | >1000 |
| amide | | | |
| Mexiletine S-methyl- | >1000 | >1000 | >1000 |
| methionine chloride | | | |
| amide | | | |

### Example 8

### Evaluation of the Systemic Availability of Mexiletine in the Dog from Various Mexiletine Prodrugs

Methods

Test substances (i.e., mexiletine, and various mexiletine amino acid prodrugs) were administered by oral gavage to groups of five dogs. The characteristics of the test animals are set out in Table 6.

TABLE 6
Characteristics of experimental dogs used in study

| | |
|---|---|
| Species | Dog |
| Type | Beagle |
| Number and sex | 5 males |
| Approximate age | 4-6 months at the start of treatment |
| Approx. bodyweight | 7-9 kg at the start of treatment |

Blood samples were taken at various times after administration and submitted to analysis for parent drug using a validated LC-MS-MS assay. Pharmacokinetic parameters derived from the plasma analytical were determined using Win Nonlin. The results are given in Table 7.

Results

The data show significant variability in the systemic availability of mexiletine from the various amino acid prodrugs tested. For example from the nicotinic and isonicotinic acid amides there was negligible bioavailability with respect to mexiletine. Conversely oral administration of the glutamine amide prodrug resulted in near complete bioavailability.

TABLE 7
Comparative pharmacokinetics of mexiletine in the dog
following oral dosing with various amino acid prodrugs
of mexiletine at 1 mg/kg mexiletine free base

| | Cmax | AUC | F (abs) | T50% |
|---|---|---|---|---|
| Compound | (ng/mL) | (ng · h/mL) | (%) | Cmax (h) |
| Mexiletine | 178 ± 35 | 1270 ± 370 | 113 ± 33 | 5.0 |
| Mexiletine | 151 ± 19 | 867 ± 135 | 85 ± 11 | 5.0 |
| Mexiletine glutamic | 104* | 1020* | 103** | 7.6* |
| acid amide | | | | |
| Mexiletine | 117* | 790* | 82** | 5.6* |
| glutamine amide | | | | |
| Mexiletine valine- | 77.2 ± 13.6 | 565 ± 168 | 50 ± 9 | 4.25 |
| valine amide | | | | |
| Mexiletine arginine | 74.3 ± 8.9 | 564 ± 164 | 50 ± 14 | 6.5 |
| amide | | | | |
| Mexiletine glycine | 79.6 ± 4.7 | 513 ± 55 | 57 ± 6 | 5.5 |
| amide | | | | |
| Mexiletine | 86.9* | 505* | 58** | 5.4* |
| homoarginine amide | | | | |
| Mexiletine | 72.5 ± 9.9 | 424 ± 61 | 38 ± 6 | 3.5 |
| phenylalanine amide | | | | |
| Mexiletine citrulline | 93.1 ± 30.3 | 411 ± 103 | 37 ± 10 | 3.0 |
| amide | | | | |
| Mexiletine valine | 69.4 ± 19.4 | 396 ± 84 | 35 ± 6 | 5.0 |
| amide | | | | |
| Mexiletine lysine | 54.4 ± 10.7 | 301 ± 55 | 27 ± 5 | 5.0 |
| amide | | | | |
| Mexiletine | 0.59* | No value | No value | No value |
| isonicotinic acid | | | | |
| amide | | | | |
| Mexiletine nicotinic | 0.00/0.00* | No value | No value | No value |
| acid amide | | | | |
| Mexiletine | 0.7* | No value | No value | No value |
| aminocyclo- | | | | |
| propylglycine | | | | |
| carboxylic acid | | | | |
| amide | | | | |

*mean of two results
**relative bioavailability in two animals

### Example 9

### Evaluation of the Systemic Availability of Mexiletine in the Cynomolgus Monkey from Various Mexiletine Prodrugs

Methods

Test substances (i.e., mexiletine, and various mexiletine amino acid prodrugs) were administered by oral gavage to groups of five male cynomolgus monkeys Blood samples were taken at various times after administration and submitted to analysis for the parent drug using a validated LC-MS-MS assay. Pharmacokinetic parameters derived from the plasma analytical were determined using Win Nonlin. The results are given in Table 8.

Results

As in the dog the data show significant variability in the systemic availability of mexiletine from the various amino acid prodrugs tested. Again from the nicotinic and isonicotinic acid amides the bioavailability with respect to mexiletine was negligible whereas the glutamine amide prodrug resulted in near complete bioavailability.

TABLE 8
comparative pharmacokinetics of mexiletine in
the monkey following oral dosing with various amino
acid prodrugs of mexiletine at 1 mg/kg mexiletine free base

| | Cmax | AUC | F (abs) | T50% |
|---|---|---|---|---|
| Compound | (ng/mL) | (ng · h/mL) | (%) | Cmax (h) |
| Mexiletine | 176 ± 25 | 857 ± 188 | 78.6 ± 14.1 | 3.11 |
| Mexiletine | 105* | 649* | 124** | 4.0* |
| glutamine amide | | | | |
| Mexiletine | 90.6* | 604* | 117** | 4.8* |
| glutamic acid | | | | |
| amide | | | | |
| Mexiletine | 86.9 ± 9.68 | 453 ± 76.1 | 41.5 ± 4.11 | 3.71 |
| glycine amide | | | | |
| Mexiletine | 56.8* | 424* | 72.9** | 5.13 |
| homoarginine | | | | |
| amide | | | | |
| Mexiletine | 1.39* | No value | No value | No value |
| isonicotinic | | | | |
| acid amide | | | | |
| Mexiletine | 1.04* | No value | No value | No value |
| nicotinic acid | | | | |
| amide | | | | |
| Mexiletine | 024* | No value | No value | No value |
| aminocyclo- | | | | |
| propylglycine | | | | |
| carboxylic acid | | | | |
| amide | | | | |

*mean of two results
**relative bioavailability in two animals

### Example 10

### Effects of Mexiletine and Mexiletine Glycine and Lysine Amides on Contractions of Rabbit Stomach Smooth Muscle

Using two prototypic amino acid conjugates of mexiletine (mexiletine glycine and lysine amides) with reduced sodium channel blocking potencies, the comparative direct effects of these vs mexiletine on rabbit stomach smooth muscle were examined. The magnitude of any such direct effects may be expected to be a determinant of the emesis associated with mexiletine. Reduction in any direct effects on EFS stimulated stomach smooth muscle may therefore be expected to result in a lesser emetic response.

Methods

Strips (˜15×2 mm) of full thickness rabbit stomach smooth muscle (mucosa intact) cut from antrum area of stomach were mounted between platinum ring electrodes. The tissue was stretched to a steady tension of about 1 g and changes in force production were recorded using sensitive transducers.

Optimal voltage for stimulation was determined while the tissue was paced with an electrical field stimulation (EFS) at 14 Hz, with a pulse width of 0.5 msec. Trains of pulses then continued for 20 seconds, every 50 seconds.

EFS at optimal voltage continued throughout the protocol (stable responses=“baseline measurement of EFS”).

The test conditions employed were as follows:

(1) vehicle (deionized water, added at equivalent volume additions to test articles),
(2) Mexiletine at 7 concentrations (10 nM, 100 nM, 1 mM, 3 mM, 10 mM, 30 mM, 100 mM),
(3) Mexiletine-lysine-amide at 7 concentrations (10 nM, 100 nM, 1 mM, 3 mM, 10 mM, 30 mM, 100 mM), and
(4) Mexiletine-glycine-amide at 7 concentrations (10 nM, 100 nM, 1 mM, 3 mM, 10 mM, 30 mM, 100 mM).

Following 10 minutes of baseline EFS, the first addition of test article or vehicle (deionized water) was performed.

Test concentrations were added in a cumulative manner with PBS washes between each addition.

Test concentrations were added in a non-cumulative manner with PSS washes between each addition. Next, TTX (Na+ channel blocker) was added to the samples to confirm EFS responses were elicited via nerve stimulation, as well as to confirm activity of a sodium channel blocker (the same mechanism as mexiletine). EFS was then stopped.

Results

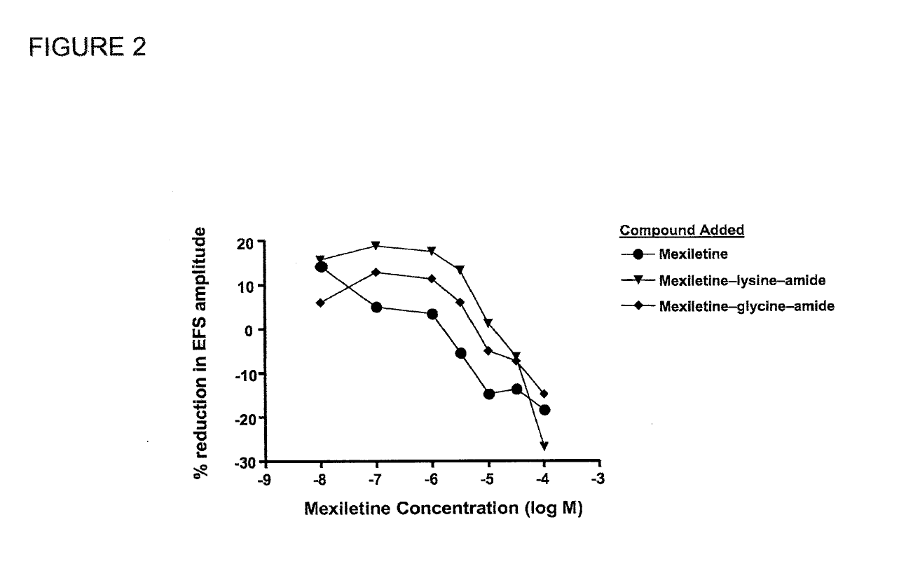

The results of this investigation are encapsulated in FIG. 2 which shows a clear difference in effects of mexiletine itself and mexiletine lysine-amide and mexiletine glycine-amide on rabbit stomach smooth muscle. While all three compounds progressively attenuated the EFS induced contractions of rabbit stomach, the prodrug conjugates were significantly less potent in doing so. The calculated ED50 values were 2.17, 9.16, and 21.83 μM for mexiletine, mexiletine glycine amide and mexiletine lysine amide respectively. The magnitude of reduction in potency in this functional assay is consistent with that observed during the in vitro assessment of blockade of the Nav1.1 channel and suggests the latter may be a good indicator of likely effects on the stomach epithelium. Such a reduction in the potential for direct actions on stomach muscle may minimize the likelihood of a directly mediated emetic response to the prodrug.

### Example 11

### Mexiletine and mexiletine-glycine-amide—Assessment of Emetic Effects Following Oral Administration to the Ferret

Using a prototypic amino acid conjugate of mexiletine with reduced sodium channel blocking potency (mexiletine-glycine-amide) the comparative emetic effects of this versus mexiletine in the ferret were examined.

Methods

Male ferrets (n=7) were allowed free access to pelleted diet until late afternoon on the day prior to the day of each test. The food was then removed and the ferrets were starved overnight. Food was not returned until after completion of the emetic observation. On the morning of the study, the animals were orally dosed with either 20 mg/kg mexiletine hydrochloride solution or a molar equivalent dose of mexiletine glycine amide, using a constant dose volume of 5 mL/kg. The animals were continuously observed for 2 hours post oral treatment and any incidences of retching and vomiting were recorded.

Results

The results presented in Tables 9 and 10 show a significantly reduced frequency and duration of emesis after giving the prodrug in comparison to that seen after administering the parent compound. The average number of vomits after prodrug administration dropped to less than 30% of those observed after dosing the parent drug. Similarly, the duration of vomiting was very much reduced after prodrug administration, to less than 30% of that seen after administering mexiletine itself. Potentially, these data show a reduced ability for this prototypic mexiletine amino acid prodrug to give rise to nausea and vomiting in man, which would be expected to lead to improved efficacy and patient compliance.

TABLE 9
Effects of mexiletine and its glycine amide prodrug on retching and
vomiting in the ferret

| | | Total number | | | |
|---|---|---|---|---|---|
| | | of individual | | Time (min) | |
| | Animal | incidences of: | | to onset of: | |
| Treatment | no. | Retching | Vomiting | Retching | Vomiting |
| Mexiletine | 1 | 46 | 12 | 19 | 19 |
| hydrochloride | 2 | 9 | 3 | 30 | 30 |
| 20 mg/kg | 3 | 37 | 9 | 16 | 16 |
| | 4 | 8 | 1 | 17 | 20 |
| | 5 | 30 | 11 | 15 | 15 |
| | 6 | 16 | 3 | 14 | 14 |
| | 7 | 26 | 3 | 17 | 18 |
| Mexiletine- | 1 | 20 | 5 | 15 | 17 |
| glycine-amide | 2 | 2 | 1 | 13 | 13 |
| 20 mg/kg | 3 | 12 | 2 | 11 | 11 |
| (molar | 4 | 0 | 0 | >120 | >120 |
| equivalent dose | 5 | 10 | 1 | 15 | 15 |
| to mexiletine | 6 | 2 | 0 | 10 | >120 |
| HCl) | 7 | 20 | 3 | 13 | 13 |

TABLE 10
Comparison of the effects of mexiletine hydrochloride and
mexiletine glycine amide on retching and vomiting in the ferret

| | Group mean of total | | Group mean of | |
|---|---|---|---|---|
| | number of individual | | duration (min) of total | |
| | incidences of (±se): | | period of (±se): | |
| Treatment | Retching | Vomiting | Retching | Vomiting |
| Mexiletine | 24.6 ± 5.42 | 6.0 ± 1.70 | 6.7 ± 1.76 | 5.0 ± 1.27 |
| hydrochloride | | | | |
| 20 mg/kg | | | | |
| Mexiletine- | 9.4* ± 3.20 | 1.7* ± 0.68 | 2.0* ± 0.90 | 1.3* ± 0.52 |
| glycine-amide | | | | |
| 20 mg/kg | | | | |

Statistical difference from mexiletine HCl
*p < 0.05 (t test)

### Example 12

### Evaluation of the Comparative Systemic Availability of Mexiletine from the Parent Drug Versus Mexiletine Glycine Amide in the Ferret

In order to confirm that the lesser emetic effect associated with the prototypic prodrug prodrug mexiletine glycine amide was not simply the consequence of lower systemic availability of the drug a comparative pharmacokinetic study was undertaken.

Methods

Test substances (i.e., mexiletine & mexiletine glycine amide) were administered by oral gavage to a group of six ferrets.

Blood samples were taken at various times after administration and submitted to analysis for the prodrug and parent drug using a validated LC-MS-MS assay. Pharmacokinetic parameters derived from the plasma analytical were determined using Win Nonlin.

Results

The results are given in Table 11. Comparing systemic exposure to the drug after giving either the drug itself or the glycine amide prodrug showed a comparable overall exposure to mexiletine. As shown in Table 11 the mean relative bioavailability of mexiletine from the glycine prodrug was 94% of that after giving the parent molecule, providing confirmation that the reduced emesis associated with the prodrug was not due to poor systemic exposure to the drug.

TABLE 11
Pharmacokinetics of mexiletine in the ferret after oral administration of 10 mg
mexiletine free base equivalents/kg of either mexiletine itself or mexiletine
glycine amide

| Pharmacokinetic | Ferret Number | | | | | | | |
|---|---|---|---|---|---|---|---|---|
| parameter | 1 | 2 | 3 | 4 | 5 | 6 | Mean | sd |

Mexiletine

| | | | | | | | | |
|---|---|---|---|---|---|---|---|---|
| Cₘₐₓ (ng/mL) | 3770 | 2890 | 3270 | 4070 | 4420 | 2820 | 3540 | 650 |
| Tₘₐₓ (h) | 0.5 | 0.5 | 0.5 | 0.5 | 0.5 | 0.5 | 0.5^{a} | |
| AUC (ng · h/mL) | 24400 | 15600 | 18700 | 22400 | 18500 | 17500 | 19500 | 3300 |
| t½ (h) | 4.0 | 3.9 | 4.6 | 4.3 | 4.6 | 6.1 | 4.5 | |

Mexiletine glycine amide

| | | | | | | | | |
|---|---|---|---|---|---|---|---|---|
| Cₘₐₓ (ng/mL) | 1890 | 1690 | 1750 | 1960 | 1840 | 1590 | 1790 | 140 |
| Tₘₐₓ (h) | 2 | 2 | 2 | 1 | 0.5 | 2 | 2^{a} | |
| AUC (ng · h/mL) | 16400 | 16700 | 19200 | 17200 | 19900 | 17800 | 17900 | 1400 |
| t½ (h) | 4.3 | 5.0 | 7.1 | 5.7 | 6.7 | 7.0 | 5.7^{b} | |
| Fᵣₑₗ (%) | 67 | 107 | 103 | 77 | 108 | 101 | 94 | |

^{a}Median value for Tₘₐₓ
^{b}Calculated as ln2/mean k

### Summary

The present invention is directed to mexiletine and p-OH mexiletine prodrugs of Formula I:

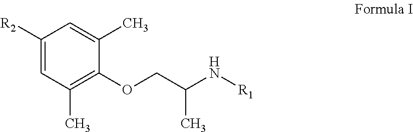

as defined above.

The present invention also relates to a composition comprising the compound of Formula (I) and a pharmaceutically acceptable excipient.

The invention also relates to a compound of Formula (I) for use in treating pain without inducing GI side effects associated with mexiletine.
The gastrointestinal side effects associated with mexiletine are nausea, dyspepsia, vomiting, diarrhea, constipation or a combination of these side effects.
The pain being treated is neuropathic pain such as that associated with diabetic neuropathy, acute and chronic nerve pain, alcoholic polyneuropathy, chronic pain from radiotherapy, thalamic pain and diabetic truncal pain, pain due to neuralgia, erythromelaglia, chronic cryptogenic sensory polyneuropathy and pain associated with cancer and its treatment.
The prodrug may be mexiletine lysine amide, mexiletine homoarginine amide, mexiletine glutamic acid amide, mexiletine glutamine amide or mexiletine methylmethionine amide.

1. A compound of Formula (I) 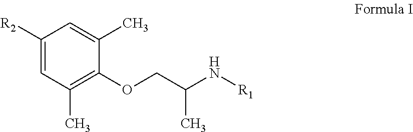
or a pharmaceutically acceptable salt thereof,
wherein,
R₁ is selected from hydrogen or 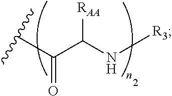
R₂ is selected from hydrogen, 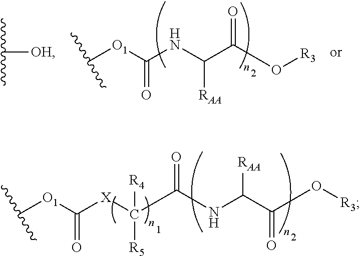
O₁ is the phenolic oxygen present in the unbound form of p-OH mexiletine;
X is (—NH—), (—O—), or absent;
each occurrence of R_{AA} is independently a natural or non-natural amino acid side chain;
n₁ is an integer selected from 0 to 16;
each occurrence of n₂ is independently an integer selected from 1 to 9;
each occurrence of R₃ is independently selected from hydrogen, or an optionally substituted alkyl group;
each occurrence of R₄ and R₅ is independently selected from hydrogen, 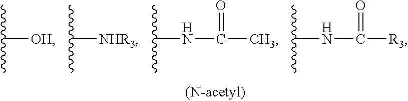
or an optionally substituted alkyl group;
wherein in the case of a double bond occurring in the carbon chain defined by n₁, R₄ is present and R₅ is absent on the carbons that form the double bond; provided that
at least R₁ is 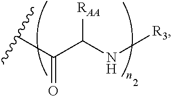
or alternatively,
at least R₂ is either 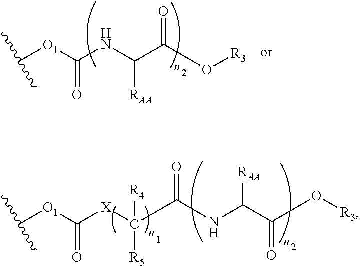
and provided that the compound is not one of the following compounds: HBr.glycine-(rac)mexiletine; AcOH.asparagine-(rac)mexiletine; TFA.tryptophan-(rac)mexiletine; HBr.alanine-(rac)mexiletine; AcOH.asparagine-(rac)mexiletine; HBr.glycine-(rac)mexiletine; AcOH-phenylalanine-(rac)mexiletine; or TFA.tryptophan-(rac)mexiletine.H₂O.

2. The compound of claim 1, wherein R¹ is hydrogen and R² is

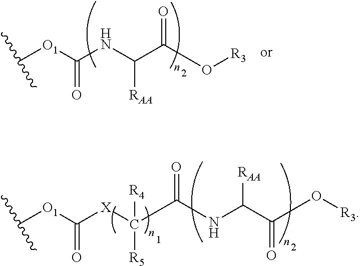

3. The compound of claim 2, wherein R² is

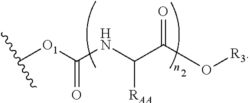

4. The compound of claim 1, wherein R² is hydrogen and R¹ is

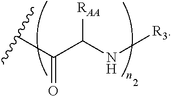

5. The compound of in any of claims 1 to 4, wherein each occurrence of R³ is hydrogen.

6. The compound of any of claims 1 to 4, wherein each occurrence of R³ is independently selected from an unsubstituted C₁₋₆ alkyl.

7. The compound of any of claims 1 to 4, wherein R⁴ is hydrogen or C₁₋₄ alkyl at each occurrence.

8. The compound of any of claims 1 to 4, wherein R⁵ is hydrogen or C₁₋₄ alkyl at each occurrence,

9. The compound of claim 8, wherein each R⁵ is hydrogen at each occurrence.

10. The compound of any of claims 1 to 4, wherein the compound of Formula (I) is not one of the following compounds:
alanine-(rac)mexiletine amide;
β-methoxy aspartic acid-(rac)mexiletine amide;
α-methoxy aspartic acid-(rac)mexiletine amide;
asparagine-(rac)mexiletine amide;
glycine-(rac)mexiletine amide;
leucine-(rac)mexiletine amide;
methionine-(rac)mexiletine amide;
(rac)methionine-(rac)mexiletine amide;
phenylalanine-(rac)mexiletine amide; or
alanine glycine glycine-(rac)mexiletine amide;
or tryptophan(rac)mexiletine.

11. The compound of any of claims 1 to 4, wherein the compound of Formula (I) has one prodrug moiety, and the prodrug moiety wherein each occurrence of n₂ is 1, 2 or 3.

12. The compound of any of claims 1 to 4, wherein each occurrence of R^{AA} is independently an amino acid side chain, wherein an amino acid residue comprises from 1 to 20 carbon atoms, or the residue is hydrogen.

13. The compound of claim 12, wherein R^{AA} is a naturally occurring amino acid.

14. The compound of claim 1, where in the compound is selected from the group consisting of:
mexiletine sarcosine amide;
mexiletine threonine amide;
mexiletine histidine amide;
mexiletine serine amide;
mexiletine 2-methyl β alanine amide;
mexiletine glycine amide;
mexiletine glutamine amide;
mexiletine cyclopropyl glycine amide;
mexiletine β amino alanine amide;
mexiletine nicotinic acid amide;
mexiletine citrulline amide;
mexiletine isonicotinic acid amide;
mexiletine homoarginine amide;
mexiletine arginine amide;
mexiletine glutamic acid amide; and
mexiletine S-methyl-methionine chloride amide.

15. A method for treating a subject with pain, arrhythmia, or a combination thereof comprising administering to a subject in need thereof an effective amount of the compound or salt according to claim 1, wherein the compound or salt exhibits a reduced adverse gastrointestinal side effect as compared to an equivalent dose of mexiletine alone.

16. The method of claim 15, wherein the gastrointestinal side effect associated with administration of mexiletine is selected from the group consisting of: emesis, nausea, and abdominal discomfort.

17. A composition comprising a compound of claim 1 and one or more pharmaceutical excipients

18. A compound of Formula (IA) 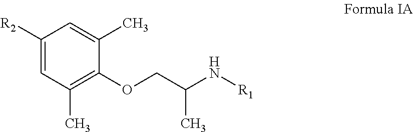
or a pharmaceutically acceptable salt thereof,
wherein,
R₁ is 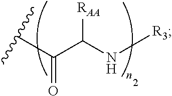
R₂ is selected from hydrogen or 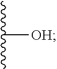
each occurrence of R_{AA} is independently a natural or non-natural amino acid side chain containing from 1 to 20 carbon atoms or hydrogen;
n₂ is an integer selected from 1, 2 or 3;
R₃ is selected from hydrogen, or an optionally substituted c₁-4 alkyl group;
provided that the compound is not one of the following compounds: HBr.glycine-(rac)mexiletine; AcOH.asparagine-(rac)mexiletine; TFA.tryptophan-(rac)mexiletine; HBr.alanine-(rac)mexiletine; AcOH.asparagine-(rac)mexiletine; HBr.glycine-(rac)mexiletine; AcOH-phenylalanine-(rac)mexiletine; or TFA.tryptophan-(rac)mexiletine.H₂O.

19. The compound of claim 18, wherein n₂ is 1.

20. The compound of claim 18 or 19 wherein R_{AA} is a natural amino acid side chain.

21. The compound of claim 18, wherein the compound is selected from the group consisting of mexiletine pipecolic amide and mexiletine dimethylglycine amide.

22. A method for treating a subject with pain, arrhythmia, or a combination thereof comprising administering to a subject in need thereof an effective amount of the compound or salt according to claim 18.
